(19) United States
(12) Patent Application Publication
(10) Pub. No.: US 2011/0046321 A1
(43) Pub. Date: Feb. 24, 2011

(54) ADDUCTS OF EPOXY RESINS DERIVED FROM ALKANOLAMIDES AND A PROCESS FOR PREPARING THE SAME

(76) Inventors: Jim D. Earls, Lake Jackson, TX (US); Robert E. Hefner, JR., Lake Jackson, TX (US)

Correspondence Address: The Dow Chemical Company, P.O. BOX 1967, 2040 Dow Center, Midland, MI, 48641, US

(21) Appl. No.: 12/989,811

(22) PCT Filed: May 18, 2009
(86) PCT No.: PCT/US09/44290

§ 371 Date: Oct. 27, 2010

Related U.S. Application Data
(60) Provisional application No. 61/055,234, filed on May 22, 2008.

(51) Int. Cl. C08L 63/00; C09D 163/00; C07D 303/46

(52) U.S. Cl. 525/523; 549/553

(57) ABSTRACT

An adduct and a process for preparing such adduct, wherein the adduct includes at least one reaction product of an epoxy resin material (A) and a compound (B); wherein the epoxy resin material (A) comprises a glycidyl ether or glycidyl ester of an alkanolamide; and compound (B) comprises a compound having two or more reactive hydrogen atoms per molecule, and the reactive hydrogen atoms are reactive with epoxide groups. A curable epoxy resin composition can be prepared from (i) the adduct described above, and (ii) one or more epoxy resins other than the epoxy resin material (A). A cured epoxy resin may be prepared from such curable composition including an article such as a coating, an electrical or structural laminate, an electrical or structural composite, a filament winding, a molding, a casting, and an encapsulation.

### FIELD OF THE INVENTION

The present invention relates generally to adducts of epoxy resins. More specifically, the present invention relates to adducts formed from glycidyl ether amides and glycidyl ester amides derived from alkanolamides, and epoxy compositions comprising the same.

### BACKGROUND OF THE DISCLOSURE

Epoxy resins are one of the most widely used engineering resins, and are well-known for their use in composites with high strength fibers. Epoxy resins form a glassy network, exhibit excellent resistance to corrosion and solvents, good adhesion, reasonably high glass transition temperatures, and adequate electrical properties. Unfortunately, crosslinked, glassy epoxy resins with relatively high glass transition temperatures (>100° C.) are brittle. The poor impact strength of high glass transition temperature epoxy resins limits the usage of epoxies as structural materials and in composites. Another major use for epoxy resins is in the preparation of coatings. While good adhesion, hardness and corrosion resistance can be achieved in said coatings, there is substantial room for improvement in the toughness and impact resistance, especially as glass transition temperature is increased. Furthermore, coatings prepared using aromatic epoxy resins suffer from chalking during exposure to sunlight. This severely limits the use of such coatings in outdoors applications.

Frischinger et al. disclose vegetable oils, which have been epoxidized through the double bonds in the backbone, used in blends with the diglycidyl ether of Bisphenol A. Frischinger, P. Muturi, S Dirlikov, Two Phase Interpenetrating Epoxy Thermosets that Contain Epoxidized Triglyceride Oils. Part II. Applications, Advances in Chemistry Series (1995), 239 (Interpenetrating Polymer Networks), 539-56.

H. Bjornberg, Novel Primary Epoxides, WO 00118751, Apr. 6, 2000, discloses that products obtained by esterifying an alcohol with an alkenoic acid may be epoxidized through the terminal double bonds and used in blends with the diglycidyl ether of Bisphenol A.

Poly(glycidyl ethers), NL 660241 1, Aug. 8, 1966 discloses that poly(glycidyl ethers) of castor oil are prepared by reaction of castor oil with epihalohydrin in the presence of a Lewis acid catalyst with formation of polyhalohydrin esters of castor oil after which the latter are dehydrohalogenated.

J. L. Cecil, W. J. Kurnik, D. E. Babcock, Coating Compositions Containing Glycidyl Ethers of Fatty Esters, U.S. Pat. No. 4,786,666, Nov. 22, 1988, disclose high-solids coating compositions based on bisphenol diglycidyl ethers, castor oil polyglycidyl ethers, bisphenols, fatty acids and dimmer acids.

S. F. Thames, H. Yu, R. Subraminian, Cationic Ultraviolet Curable Coatings from Castor Oil, Journal of Applied Polymer Science (2000), 77(1), 8-13, disclose coatings formulated from castor oil glycidyl ether, epoxy resin UVR 6100, and photoinitiator UVI 6990.

Conventional epoxy resin adducts, and their preparation, have been described in various references. For example, an adduct of diethylenetriamine and diglycidyl ether of bisphenol A is described by Henry Lee and Kris Neville in Handbook of Epoxy Resins published by McGraw Hill, Inc., New York, (1967) on pages 7-15 to 7-19. D.E.H.™ 52 (manufactured and marketed by The Dow Chemical Company, Midland, Mich.) is a commercial adduct product of diethylenetriamine and diglycidyl ether of bisphenol A.

Daniel A. Scola in Developments in Reinforced Plastics 4 published by Elsevier Applied Science Publishers Ltd., England, pages 196-206 (1984) describes amine adducts of epoxy resins. The epoxy resin is selected from the diglycidyl ether of bisphenol A, tetraglycidyl 4,4′-diaminodiphenylmethane, triglycidyl p-aminophenol, epoxy phenol or cresol novalacs, hydrogenated diglycidyl ether of bisphenol A, or any combination thereof. The amine may be aliphatic, cycloaliphatic, aromatic or alkylaromatic diamine.

J. Klee, et al. in Crosslinked Epoxies published by Walter de Gruyter and Co., Berlin, pages 47-54 (1987) describes the synthesis and analytical characterization of adducts of the diglycidyl ether of bisphenol A with primary monoamines including aniline, p-chloroaniline, benzylamine and cyclohexylamine.

Typical performance requirements of thermoset resins, including epoxy resins, include a high softening point (>200° C.), low flammability, hydrolytic resistance, chemical and solvent resistance, and a stable dielectric with temperature variations. Epoxy resins may provide these properties, but various epoxy systems may include the drawback of slow hardening cycles due to slow kinetics.

Other drawbacks to various epoxy systems are the use of solvents, the resulting reaction by-products, and/or insufficient UV stability. Solvents and reaction by-products may result in unwanted chemical exposure or release and bubble formation during cure. Insufficient UV stability may also limit the end uses of epoxy systems, completely preventing their use in most outdoor applications.

Accordingly, there exists a need for improvements in the processing of epoxy resins, such as by lowering viscosities and eliminating the need for solvents. There also exists a need to improve the performance of epoxy resin coatings, such as improvements in UV stability and flexibility and damage tolerance.

### SUMMARY OF THE INVENTION

In one aspect, embodiments disclosed herein relate to an adduct comprising at least one reaction product of an epoxy resin material (A) and a compound (B); wherein the epoxy resin material (A) comprises a glycidyl ether or glycidyl ester of an alkanolamide; and wherein compound (B) comprises a compound having two or more reactive hydrogen atoms per molecule, and the reactive hydrogen atoms are reactive with epoxide groups.

In another aspect, embodiments disclosed herein relate to an adduct comprising at least one reaction product of an epoxy resin material (A), a compound (B) and an epoxy resin compound (C); wherein the epoxy resin material (A) comprises a glycidyl ether or glycidyl ester of an alkanolamide; wherein the compound (B) comprises a compound having two or more reactive hydrogen atoms per molecule, the reactive hydrogen atoms are reactive with epoxide groups, and wherein the resin compound (C) comprises one or more epoxy resins other than the epoxy resin material (A).

In still another aspect, embodiments disclosed herein relate to a process for preparing the above adducts.

In yet another aspect, embodiments disclosed herein relate to curable epoxy resin compositions comprising the above adducts and a resin compound (D); wherein the resin compound (D) comprises one or more epoxy resins other than the epoxy resin material (A) and other than the epoxy resin (C).

In still another aspect, embodiments disclosed herein relate to a process for preparing the above curable epoxy resin compositions.

In another aspect, embodiments disclosed herein relate to a cured epoxy resin prepared by the above process.

In yet another aspect, embodiments disclosed herein relate to an article comprising the above cured epoxy resin.

Other aspects and advantages of the present invention will be apparent from the following description and the appended claims.

### DETAILED DESCRIPTION OF THE PREFERRED EMBODIMENTS

In one aspect, embodiments disclosed herein relate to improvements in the processing and performance of epoxy resin coatings. More specifically, embodiments disclosed herein relate to new adducts of glycidyl ethers and glycidyl esters derived from alkanolamides. These glycidyl ethers and glycidyl esters may be used in combination with other epoxy resins, and may result in improved processing, UV stability, and flexibility/damage tolerance of the resulting epoxy resin coatings, composites, adhesives, electronics, and molded articles.

As noted above, the adduct of the present invention may include at least one reaction product of an epoxy resin material (A) and a compound (B), wherein the epoxy resin material (A) comprises a glycidyl ether amide or glycidyl ester amide derived from an alkanolamide, and wherein the compound (B) comprises a compound having two or more reactive hydrogen atoms per molecule, and the reactive hydrogen atoms are reactive with epoxide groups.

As used herein, the term “adduct” means a product of a direct addition of two or more distinct molecules, resulting in a single reaction product. The resultant product or adduct is considered a distinct molecular species from the reactants.

For the epoxy resin material (A) useful in the present invention, the adducts of the present invention utilize epoxy resins based on seed oil alkanolamides, for example, those seed oil based alkanolamides described in co-pending U.S. patent application Ser. No. ______ (Attorney Docket No. 65426) incorporated herein by reference. In another embodiment, the adducts of the present invention utilize epoxy resins based on non-seed oil alkanolamides, for example, those non-seed oil based alkanolamides described in co-pending U.S. patent application Ser. No. ______ (Attorney Docket Nos. 65843) incorporated herein by reference.

It has been discovered, as disclosed in co-pending U.S. patent application Ser. Nos. ______ and ______ (Attorney Docket Nos. 65426 and 65843, respectively), each of which are incorporated herein by reference, that epoxy resins comprising glycidyl ether amides and glycidyl ester amides derived from alkanolamides have improved properties and may be formed by an advantageous process described in the above co-pending patent applications. Said process for forming the epoxy resins useful as epoxy resin material (A) of the present invention results in avoidance of hydrolysis of any amide linkages that are present and epoxidation achieving at least 80% or more of theoretical. As a result of such process, epoxy resins comprising the glycidyl ethers and glycidyl esters derived from alkanolamides as disclosed in the above co-pending patent applications may have very low chloride (including ionic, hydrolyzable and total chloride) content and high diglycidyl ether content, which may provide the epoxy resins with increased reactivity toward conventional epoxy resin curing agents, reduced potential corrosivity, improved mechanical properties, and improved electrical properties.

Embodiments disclosed herein use the epoxy resins comprising the glycidyl ether amides and glycidyl ester amides derived from alkanolamides to react with compounds comprising two or more reactive hydrogen atoms per molecule to produce adducts. These adducts can be blended with one or more epoxy resins and, optionally, with a curing agent and/or a catalyst to form a curable epoxy resin composition. By curing the curable epoxy resin composition, a cured epoxy resin can be obtained.

In one aspect, embodiments disclosed herein relate to improvements in the processing and performance of epoxy resin coatings. More specifically, embodiments disclosed herein relate to new adducts of glycidyl ethers and glycidyl esters derived from alkanolamides. These glycidyl ethers and glycidyl esters may be used in combination with other epoxy resins, and may result in improved processing, UV stability, and flexibility/damage tolerance of the resulting epoxy resin coatings, composites, adhesives, electronics, and molded articles.

As an example of the epoxy resin material (A) useful in the present invention, the epoxy resins comprising the glycidyl ether amides and glycidyl ester amides derived from alkanolamides may be used in the present invention including for example, the glycidyl ether amides and glycidyl ester amides s derived from fatty acid esters, fatty acids and fatty acid triglycerides (A) as may be represented by Formula I:

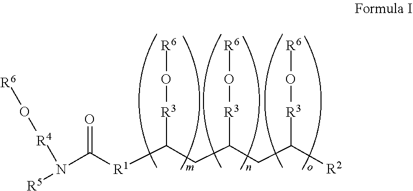

wherein R¹ and R⁴ are polyvalent hydrocarbylene moieties; R² is hydrogen or a monovalent alkyl moiety; R³ is nil or a polyvalent hydrocarbylene moiety; R⁵ is H, a monovalent alkyl moiety, or a moiety represented by Formula II:

—R⁴—O—R⁶ Formula II

wherein R⁴ is as defined above and R⁶ is a moiety of either Formula III or Formula IV:

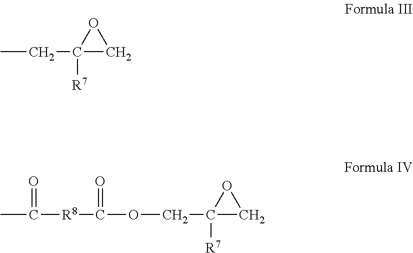

wherein R⁷ is hydrogen or an aliphatic hydrocarbon group having from 1 to about 4 carbon atoms; R⁸ is a polyvalent hydrocarbylene moiety; and m, n, and o are independently 0 or 1, provided, however, that a sum of m, n and o is a positive integer greater than zero.

An additional aspect of the present invention comprises the above glycidyl ether amides and glycidyl ester amides in admixture with glycidyl ether amides and glycidyl ester amides represented by Formula I wherein the sum of m, n and o is zero. These compositions preferably contain greater than or equal to 70 percent by weight (wt %), more preferably greater than 90 wt %, in each case based upon total composition weight, of the glycidyl ether amides and glycidyl ester amides having a sum of m, n and o greater than zero.

In other embodiments, the epoxy resin material (A) useful for preparing the epoxy resin adducts of the present invention disclosed herein may be one or more of the glycidyl ether amides and glycidyl ester amides derived from alkanolamides such as the epoxy resins comprising, for example, the glycidyl ether amides and glycidyl ester amides derived from alkanolamides as may be represented by Formula V:

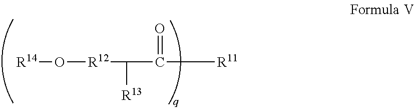

wherein R¹¹ is a mono or polyvalent hydrocarbylene moiety; R¹² is a polyvalent hydrocarbylene moiety; R¹³ is H, a monovalent alkyl moiety, or a moiety represented by Formula VI:

—R¹²—O—R¹⁴ Formula VI

wherein R¹² is as defined above and R¹⁴ is a moiety of either Formula VII or Formula VIII:

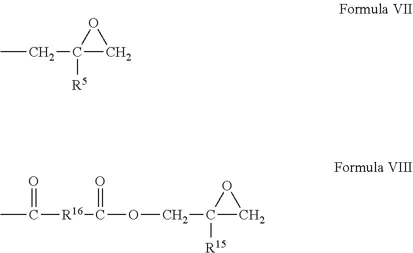

wherein R¹⁵ is hydrogen or an aliphatic hydrocarbon group having from 1 to about 4 carbon atoms; R¹⁶ is a polyvalent hydrocarbylene moiety; and q is an integer from 1 to 4.

In some embodiments, glycidyl ether and ester amide compositions disclosed herein may additionally include one or more of the following: monoglycidyl ethers or esters derived from alkanolamides; oligomers of the glycidyl ethers or esters derived from alkanolamides; and combinations thereof.

In general, the glycidyl ether and glycidyl ester amides, useful as epoxy resin material (A) of the present invention described herein may be prepared by a process (for example, an epoxidation reaction process) comprising reacting (a) a OH or COOH functionalized alkanolamide or mixtures thereof with (b) an epihalohydrin, and (c) a basic acting substance in a solid form. The process may optionally comprise any one or more of the following components: (d) a solvent, (e) a catalyst and/or (f) a dehydrating agent.

Functionalized saturated fatty acid esters, fatty acids and fatty acid triglycerides may be formed, for example, by the aminolysis of a fatty acid ester, fatty acids or fatty acid triglyceride, such as disclosed in co-pending U.S. patent application Ser. No. ______ (Attorney Docket No. 65426), which is incorporated herein by reference. Aminolysis may include reaction of fatty acid esters or fatty acid triglycerides with amino diols and triols, such as diethanolamine, triethanolamine, triisopropanolamine, tri[(2-hydroxy)1-propyl]amine, 2-amino-2-methyl-1,3-propanediol, 2-amino-2-hydroxymethyl 1,3-propanediol, 2-amino-2-methyl ethanol and the like.

The process for preparing the glycidyl ether and esters (A) involves an initial reaction of a functionalized alkanolamide fatty acid ester or fatty acid triglyceride with an epihalohydrin to form a halohydrin intermediate. The halohydrin intermediate is then reacted with basic acting substance to convert said halohydrin intermediate to the epoxy resin final product (the glycidyl ether and/or glycidyl ester epoxy resins). If an alkali metal or alkaline earth metal hydroxide is used as a catalyst and is employed in stoichiometric or greater quantities, the initial reaction of the functionalized alkanolamide fatty acid ester or fatty acid triglyceride and the epihalohydrin produces the halohydrin intermediate in situ and the halohydrin intermediate is then converted to the epoxy resin final product without the addition of the basic acting substance.

Representative glycidyl ethers and esters from functionalized alkanolamide fatty acid esters, fatty acids and fatty acid triglycerides represented by Formula I and glycidyl ethers or esters of alkanolamides represented by Formula V include those given by the following formulae:

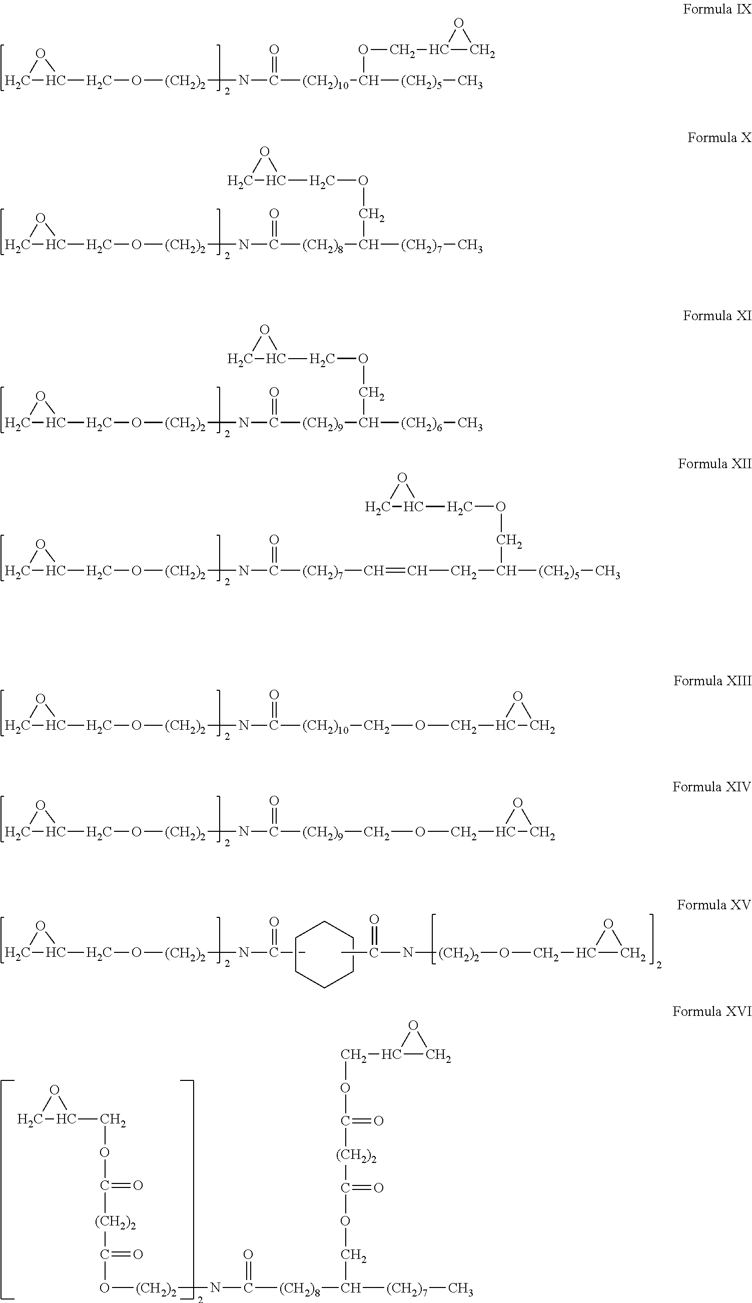

Other components may be present in minor amounts in the glycidyl ether amides and glycidyl ester amides (A). The amounts and types of the minor components may vary depending on the specific chemistry of the components present in the glycidyl ethers and esters (A) and the process used to prepare the glycidyl ethers and esters (A). In general, the glycidyl ethers and esters (A) may comprise from trace amount to less than about 40 percent, preferably from trace amount to about 5 percent of the minor components based on the total weight of the glycidyl ethers and esters (A). Examples of the minor components may include monoglycidyl ether or ester, diglycidyl ether or ester, oligomers, chlorohydrin intermediate, and any combination thereof. By “trace amounts” it is meant an amount that can be measured and limit of detection by routine analytical methods, such as, for example gas chromatographic analysis, high pressure liquid chromatographic analysis, or gel permeation chromatographic analysis.

Alcoholysis of fatty acid triglycerides can be used to form the methyl esters to be used for aminolysis or the aminolysis can be conducted on the fatty acid triglycerides directly without going through the alcoholysis step.

The glycidyl ethers or esters from alkanolamides represented by Formula V may be varied to advantageously influence the ultimate properties of the adduct compositions described herein. For example, reactants higher in monoglycidyl ether or diglycidyl ether and/or free of or low in oligomers generally favor a liquid and lower viscosity adduct or a lower melting point adduct, but possess reduced functionality (fewer active hydrogen atoms reactive with an epoxide group). Conversely, reactants lower in monoglycidyl ether or diglycidyl ether and/or higher in oligomers generally favor a higher viscosity adduct or a higher melting point adduct, with increased functionality.

Additionally, variation of the amount of monoglycidyl ether or diglycidyl ether present may be used to influence the properties of the reactant used in the process to prepare the adduct compositions in addition to the aforementioned properties of the adduct compositions. For example, a higher monoglycidyl ether or diglycidyl ether content favors reduction in the functionality of the epoxy resin reactant used in the process to prepare the adduct product.

As aforementioned, adducts of the present invention may be formed by reacting the above described glycidyl ethers and esters (A) with a compound (B), which includes compounds having two or more reactive hydrogen atoms per molecule, and the reactive hydrogen atoms are reactive with epoxide groups.

Compound (B) may include one or more compounds containing two or more hydrogens which are reactive with an epoxide group, which may include one or more of the following compounds: (a) di- and polyphenols; (b) di- and polycarboxylic acids; (c) di- and polymercaptans; (d) di- and polyamines; (e) primary monoamines; (f) sulfonamides; (g) aminophenols; (h) aminocarboxylic acids; (i) phenolic hydroxyl containing carboxylic acids; (j) sulfanilamides; and mixtures thereof.

Compound (B), useful in the embodiments of the present invention disclosed herein, is used to react with the glycidyl ethers and glycidyl esters, epoxy resin material (A), to form the adducts of the present invention. Compound (B) comprises at least one compound having two or more reactive hydrogen atoms per molecule. The reactive hydrogen atoms are reactive with epoxide groups, such as those epoxide groups contained in the glycidyl ethers and esters of component (A).

The term “reactive hydrogen atom” as used herein means that the hydrogen atom is reactive with an epoxide group. The reactive hydrogen atom differs from other hydrogen atoms including those hydrogen atoms which are non-reactive with epoxide groups in the reaction of forming the adduct but may be reactive with epoxide groups in a later process of curing the adduct with one or more epoxy resins.

Hydrogen atoms can be non-reactive with the epoxide groups in the process of forming the adduct but reactive in a later process of curing the adduct with the epoxy resin, when there are other functional groups, which are much more reactive with the epoxide groups under reaction conditions used, present in the reaction of forming the adduct. For example, a compound (B) may have two different functional groups each bearing at least one reactive hydrogen atom, with one functional group being inherently more reactive with an epoxide group than the other under the reaction conditions used. These reaction conditions may include the use of a catalyst which favors a reaction of the reactive hydrogen atom(s) of one functional group with an epoxide group over a reaction of the reactive hydrogen atom(s) of the other functional group with an epoxide group.

Other non-reactive hydrogen atoms may also include hydrogen atoms in the secondary hydroxyl groups which formed during an epoxide ring opening reaction in the process of producing the adduct.

The compound (B) comprising at least one compound having two or more reactive hydrogen atoms per molecule may further comprise aliphatic, cycloaliphatic or aromatic groups within the compound (B) structure. The aliphatic groups may be branched or unbranched. The aliphatic or cycloaliphatic groups may also be saturated or unsaturated and may comprise one or more substituents which are inert (not reactive) to the process of preparing the adduct of the present invention including the reactants and the products. The substituents may be attached to a terminal carbon atom or may be between two carbon atoms, depending on the chemical structures of the substituents. Examples of such inert substituents include halogen atoms, preferably chlorine or bromine, nitrile, nitro, alkyloxy, keto, ether (—O—), thioether (—S—), or tertiary amine. The aromatic ring, if present within the compound (B) structure, may comprise one or more heteroatoms such as N, O, S and the like.

Examples of the compound (B) may include compounds such as (a) di- and polyphenols, (b) di- and polycarboxylic acids, (c) di- and polymercaptans, (d) di- and polyamines, (e) primary monoamines, (f) sulfonamides, (g) aminophenols, (h) aminocarboxylic acids, (i) phenolic hydroxyl containing carboxylic acids, (j) sulfanilamides, and (k) any combination of any two or more of such compounds or the like.

Examples of the di- and polyphenols include 1,2-dihydroxybenzene (catechol), 1,3-dihydroxybenzene (resorcinol), 1,4-dihydroxybenzene (hydroquinone), 4,4′-isopropylidenediphenol (bisphenol A), 4,4′-dihydroxydiphenylmethane, 3,3′,5,5′-tetrabromobisphenol A, 4,4′-thiodiphenol, 4,4′-sulfonyldiphenol, 2,2′-sulfonyldiphenol, 4,4′-dihydroxydiphenyl oxide, 4,4′-dihydroxybenzophenone, 1,1′-bis(4-hydroxyphenyl)-1-phenylethane, 3,3′,5,5′-tetrachlorobisphenol A, 3,3′-dimethoxybisphenol A, 3,3′,5,5′-tetramethyl-4,4′-dihydroxydiphenyl, 4,4′-dihydroxybiphenyl, 4,4′-dihydroxy-alpha-methylstilbene, 4,4′-dihydroxybenzanilide, 4,4′-dihydroxystilbene, 4,4′-dihydroxy-alpha-cyanostilbene, 1,1-bis(4-hydroxyphenyl)cyclohexane, 1,4-dihydroxy-3,6-dimethylbenzene, 1,4-dihydroxy-3,6-dimethoxybenzene, 1,4-dihydroxy-2-tert-butylbenzene, 1,4-dihydroxy-2-bromo-5-methylbenzene, 1,3-dihydroxy-4-nitrophenol, 1,3-dihydroxy-4-cyanophenol, tris(hydroxyphenyl)methane, dicyclopentadiene or an oligomer thereof and phenol or substituted phenol condensation products, and any mixture thereof.

Examples of the di- and polycarboxylic acids include 4,4′-dicarboxydiphenylmethane, terephthalic acid, isophthalic acid, 1,4-cyclohexanedicarboxylic acid, 1,6-hexanedicarboxylic acid, 1,4-butanedicarboxylic acid, dicyclopentadienedicarboxylic acid, tris(carboxyphenyl)methane, 1,1-bis(4-carboxyphenyl)cyclohexane, 3,3′,5,5′-tetramethyl-4,4′-dicarboxydiphenyl, 4,4′-dicarboxy-alpha-methylstilbene, 1,4-bis(4-carboxyphenyl)-trans-cyclohexane, 1,1′-bis(4-carboxyphenyl)cyclohexane, 1,3-dicarboxy-4-methylbenzene, 1,3-dicarboxy-4-methoxybenzene, 1,3-dicarboxy-4-bromobenzene, and any combination thereof.

Examples of the di- and polymercaptans include 1,3-benzenedithiol, 1,4-benzenedithiol, 4,4′-dimercaptodiphenylmethane, 4,4′-dimercaptodiphenyl oxide, 4,4′-dimercapto-alpha-methylstilbene, 3,3′,5,5′-tetramethyl-4,4′-dimercaptodiphenyl, 1,4-cyclohexanedithiol, 1,6-hexanedithiol, 2,2′-dimercaptodiethylether, 1,2-dimercaptopropane, bis(2-mercaptoethyl)sulfide, tris(mercaptophenyl)methane, 1,1-bis(4-mercaptophenyl)cyclohexane, and any combination thereof.

Examples of the di- and polyamines include 1,2-diaminobenzene, isophorone diamine, 1,3-diaminobenzene, 1,4-diaminobenzene, 4,4′-diaminodiphenylmethane, 4,4′-diaminodiphenylsulfone, 2,2′-diaminodiphenylsulfone, 4,4′-diaminodiphenyl oxide, 3,3′,5,5′-tetramethyl-4,4′-diaminodiphenyl, 3,3′-dimethyl-4,4′-diaminodiphenyl, 4,4′-diamino-alpha-methylstilbene, 4,4′-diaminobenzanilide, 4,4′-diaminostilbene, 1,4-bis(4-aminophenyl)-trans-cyclohexane, 1,1-bis(4-aminophenyl)cyclohexane, tris(aminophenyl)methane, 1,4-cyclohexanediamine, 1,6-hexanediamine, piperazine, ethylenediamine, diethyletriamine, triethylenetetramine, tetraethylenepentamine, 1-(2-aminoethyl)piperazine, bis(aminopropyl)ether, bis(aminopropyl)sulfide, bis(aminomethyl)norbornane, 2,2′-bis(4-aminocyclohexyl)propane, and any combination thereof.

Examples of the primary monoamines include aniline, 4-chloroaniline, 4-methylaniline, 4-methoxyaniline, 4-cyanoaniline, 2,6-dimethylaniline, 4-aminodiphenyl oxide, 4-aminodiphenylmethane, 4-aminodiphenylsulfide, 4-aminobenzophenone, 4-aminodiphenyl, 4-aminostilbene, 4-amino-alpha-methylstilbene, methylamine, 4-amino-4′-nitrostilbene, n-hexylamine, cyclohexylamine, aminonorbornane, and any combination thereof.

The primary monoamines represent a special class of the compound (B) of the present disclosure. According to embodiments disclosed herein, the reaction of the primary monoamine with the glycidyl ethers and esters (A) produces an adduct which is substantially difunctional (i.e., the adduct has a functionality of about 2) with respect to the reactive hydrogen atom(s) (for example, the amine hydrogen atom when the primary monoamine is used as the compound (B)) present in the adduct. When this adduct is used to cure an epoxy resin, the adduct functions as a linear chain extender. As a result, the adduct provides a linear chain extension to the epoxy resin structure instead of crosslinking the epoxy resin structure. Other classes of compound (B), such as secondary diamines, may also be used to form an adduct which may be used as a linear chain extender for curing an epoxy resin.

Ammonia represents another special class of compound (B) of the present invention. The ammonia may be used in the form of liquified ammonia (NH₃) or ammonium hydroxide (NH₄OH). If ammonium hydroxide is used, it is frequently of value to employ a solvent which improves solubility of the ammonium hydroxide and the glycidyl ether and ester epoxy resin (A) in the reaction mixture.

Examples of the sulfonamides include phenylsulfonamide, 4-methoxyphenylsulfonamide, 4-chlorophenylsulfonamide, 4-bromophenylsulfonamide, 4-methylsulfonamide, 4-cyanosulfonamide, 2,6-dimethyphenylsulfonamide, 4-sulfonamidodiphenyl oxide, 4-sulfonamidodiphenylmethane, 4-sulfonamidobenzophenone, 4-sulfonylamidodiphenyl, 4-sulfonamidostilbene, 4-sulfonamido-alpha-methylstilbene, and any combination thereof.

Examples of the aminophenols include o-aminophenol, m-aminophenol, p-aminophenol, 2-methoxy-4-hydroxyaniline, 3,5-dimethyl-4-hydroxyaniline, 3-cyclohexyl-4-hydroxyaniline, 2,6-dibromo-4-hydroxyaniline, 5-butyl-4-hydroxyaniline, 3-phenyl-4-hydroxyaniline, 4-(1-(3-aminophenyl)-1-methylethyl)phenol, 4-(1-(4-aminophenyl)ethyl)phenol, 4-(4-aminophenoxy)phenol, 4-((4-aminophenyl)thio)phenol, (4-aminophenyl)(4-hydroxyphenyl)methanone, 4-((4-aminophenyl) sulfonyl)phenol, 4-(1-(4-amino-3,5-dibromophenyl)-1-methylethyl)-2,6-dibromophenol, N-methyl-p-aminophenol, 4-amino-4′-hydroxy-alpha-methylstilbene, 4-hydroxy-4′-amino-alpha-methylstilbene, and any combination thereof.

Examples of the aminocarboxylic acids include 2-aminobenzoic acid, 3-aminobenzoic acid, 4-aminobenzoic acid, 2-methoxy-4-aminobenzoic acid, 3,5-dimethyl-4-aminobenzoic acid, 3-cyclohexyl-4-aminobenzoic acid, 2,6-dibromo-4-aminobenzoic acid, 5-butyl-4-aminobenzoic acid, 3-phenyl-4-aminobenzoic acid, 4-(1-(3-aminophenyl)-1-methylethyl)benzoic acid, 4-(1-(4-aminophenyl)ethyl)benzoic acid, 4-(4-aminophenoxy)benzoic acid, 4-((4-aminophenyl)thio)benzoic acid, (4-aminophenyl)(4-carboxyphenyl)methanone, 4-((4-aminophenyl)sulfonyl)benzoic acid, 4-(1-(4-amino-3,5-dibromophenyl)-1-methylethyl)-2,6-dibromobenzoic acid, N-methyl-4-aminobenzoic acid, 4-amino-4′-carboxy-alpha-methylstilbene, 4-carboxy-4′-amino-alpha-methylstilbene, glycine, N-methylglycine, 4-aminocyclohexanecarboxylic acid, 4-aminohexanoic acid, 4-piperidinecarboxylic acid, 5-aminophthalic acid, and any combination thereof.

Examples of the phenolic hydroxyl containing carboxylic acids include 2-hydroxybenzoic acid, 3-hydroxybenzoic acid, 4-hydroxybenzoic acid, 2-methoxy-4-hydroxybenzoic acid, 3,5-dimethyl-4-hydroxybenzoic acid, 3-cyclohexyl-4-hydroxybenzoic acid, 2,6-dibromo-4-hydroxybenzoic acid, 5-butyl-4-hydroxybenzoic acid, 3-phenyl-4-hydroxybenzoic acid, 4-(1-(3-hydroxyphenyl)-1-methylethyl)benzoic acid, 4-(1-(4-hydroxyphenyl)ethyl)benzoic acid, 4-(4-hydroxyphenoxy)benzoic acid, 4-((4-hydroxyphenyl)thio)benzoic acid, (4-hydroxyphenyl)(4-carboxyphenyl)methanone, 4-((4-hydroxyphenyl)sulfonyl)benzoic acid, 4-(1-(4-hydroxy-3,5-dibromophenyl)-1-methylethyl)-2,6-dibromobenzoic acid, 4-hydroxy-4′-carboxy-alpha-methylstilbene, 4-carboxy-4′-hydroxy-alpha-methylstilbene, 2-hydroxyphenylacetic acid, 3-hydroxyphenylacetic acid, 4-hydroxyphenylacetic acid, 4-hydroxyphenyl-2-cyclohexanecarboxylic acid, 4-hydroxyphenoxy-2-propanoic acid, and any combination thereof.

Examples of the sulfanilamides include o-sulfanilamide, m-sulfanilamide, p-sulfanilamide, 2-methoxy-4-aminobenzoic acid, 2,6-dimethyl-4-sulfonamido-1-aminobenzene, 3-methyl-4-sulfonamido-1-aminobenzene, 5-methyl-3-sulfonamido-1-aminobenzene, 3-phenyl-4-sulfonamido-1-aminobenzene, 4-(1-(3-sulfonamidophenyl)-1-methylethyl)aniline, 4-(1-(4-sulfonamidophenyl)ethyl)aniline, 4-(4-sulfonamidophenoxy)aniline, 4-((4-sulfonamidophenyl)thio)aniline, (4-sulfonamidophenyl)(4-aminophenyl)methanone, 4-((4-sulfonamidophenyl)sulfonyl)aniline, 4-(1-(4-sulfonamido-3,5-dibromophenyl)-1-methylethyl)-2,6-dibromoaniline, 4-sulfonamido-1-N-methylaminobenzene, 4-amino-4′-sulfonamido-alpha-methylstilbene, 4-sulfonamido-4′-amino-alpha-methylstilbene, and any combination thereof.

The compounds containing two or more hydrogens, useful as Compound (B), may be used in an amount sufficient to provide from about 2:1 to about 100:1 equivalents of hydrogen reactive with an epoxide group (excluding secondary hydroxyl groups formed by epoxide ring opening reaction to form the adduct) per equivalent of epoxide. In other embodiments, compounds containing two or more hydrogens may be used in an amount sufficient to provide from about 3:1 to about 60:1 equivalents of hydrogen reactive with an epoxide group (excluding secondary hydroxyl groups formed by epoxide ring opening reaction to form the adduct) per equivalent of epoxide; and from about 4:1 to about 40:1 equivalents in yet other embodiments.

The process for preparing an adduct comprises reacting at least one of an epoxy resin material (A) and a compound (B), wherein the epoxy resin material (A) comprises a glycidyl ether or ester of an alkanolamide based on at least one of a fatty acid ester, fatty acid and a fatty acid triglyceride, and the compound (B) comprises a compound having two or more reactive hydrogen atoms per molecule, and the reactive hydrogen atoms are reactive with epoxide groups.

The process may be conducted at a temperature of generally from about 0° C. to about 260° C.; preferably from about 20° C. to about 200° C.; and more preferably from about 35° C. to about 160° C.

The process may be completed generally in about 5 minutes to about one week; preferably in about 30 minutes to about 72 hours; and most preferably in about 60 minutes to about 48 hours.

In other embodiments of the present invention, adduct compositions may be formed by reacting (A) glycidyl ether and ester amide compositions derived from alkanolamides, which may additionally include monoglycidyl ethers and esters, and/or oligomers, and (C) one or more epoxy resins, with (B) one or more of the above-described compounds containing two or more hydrogens which are reactive with an epoxide group.

According to embodiments disclosed herein, the adducts may comprise at least one reaction product of the glycidyl ethers and esters (A), the compound (B), and, additionally, an epoxy resin compound (C), wherein the resin compound (C) comprises one or more epoxy resins other than the epoxy resin material (A).

The epoxy resin which can be used as the resin compound (C) may be any epoxide-containing compound which has an average of more than one epoxide group per molecule. The epoxide group can be attached to any oxygen, sulfur or nitrogen atom or the single bonded oxygen atom attached to the carbon atom of a —CO—O— group. The oxygen, sulfur, nitrogen atom, or the carbon atom of the —CO—O— group may be attached to an aliphatic, cycloaliphatic, polycycloaliphatic or aromatic hydrocarbon group. The aliphatic, cycloaliphatic, polycycloaliphatic or aromatic hydrocarbon group can be substituted with any inert substituents including, but not limited to, halogen atoms, preferably fluorine, bromine or chlorine; nitro groups; or the groups can be attached to the terminal carbon atoms of a compound containing an average of more than one —(O—CHR^{a}—CHR^{a})ₜ— group, wherein each R^{a} is independently a hydrogen atom or an alkyl or haloalkyl group containing from one to two carbon atoms, with the proviso that only one R^{a} group can be a haloalkyl group, and t has a value from one to about 100, preferably from one to about 20, more preferably from one to about 10, and most preferably from one to about 5.

More specific examples of the epoxy resin which can be used as the resin compound (C) include diglycidyl ethers of 1,2-dihydroxybenzene (catechol); 1,3-dihydroxybenzene (resorcinol), 1,4-dihydroxybenzene (hydroquinone), 4,4′-isopropylidenediphenol (bisphenol A), hydrogenated bisphenol A, 4,4′-dihydroxydiphenylmethane, 3,3′,5,5′-tetrabromobisphenol A, 4,4′-thiodiphenol; 4,4′-sulfonyldiphenol; 2,2′-sulfonyldiphenol; 4,4′-dihydroxydiphenyl oxide; 4,4′-dihydroxybenzophenone; 1,1′-bis(4-hydroxyphenyl)-1-phenylethane; 3,3′-5,5′-tetrachlorobisphenol A; 3,3′-dimethoxybisphenol A; 4,4′-dihydroxybiphenyl; 4,4′-dihydroxy-α-methylstilbene; 4,4′-dihydroxybenzanilide; 4,4′-dihydroxystilbene; 4,4′-dihydroxy-α-cyanostilbene; N,N′-bis(4-hydroxyphenyl)terephthalamide; 4,4′-dihydroxyazobenzene; 4,4′-dihydroxy-2,2′-dimethylazoxybenzene; 4,4′-dihydroxydiphenylacetylene; 4,4′-dihydroxychalcone; 4-hydroxyphenyl-4-hydroxybenzoate; 1,4-butanediol; 1,6-hexanediol; 1,4-cyclohexanedimethanol; 1,3-cyclohexanedimethanol; dipropylene glycol; poly(propylene glycol); thiodiglycol; the triglycidyl ether of tris(hydroxyphenyl)methane; the polyglycidyl ethers of a phenol or alkyl or halogen substituted phenol-aldehyde acid catalyzed condensation product (novolac resins); the tetraglycidyl amines of 4,4′-diaminodiphenylmethane; 4,4′-diaminostilbene; N,N′-dimethyl-4,4′-diaminostilbene; 4,4′-diaminobenzanilide; 4,4′-diaminobiphenyl; the polyglycidyl ether of the condensation product of a dicyclopentadiene or an oligomer thereof and a phenol or alkyl or halogen substituted phenol; and any combination thereof.

The epoxy resin which may be used as the resin compound (C) may also include an advancement reaction product of an epoxy resin with aromatic di- and polyhydroxyl or carboxylic acid containing compound. The epoxy resin used for reacting with the aromatic di- and polyhydroxyl or carboxylic acid containing compound may include the aforesaid epoxy resin comprising the di- or polyglycidyl ether.

Examples of the aromatic di- and polyhydroxyl or carboxylic acid containing compound include hydroquinone, resorcinol, catechol, 2,4-dimethylresorcinol; 4-chlororesorcinol; tetramethylhydroquinone; bisphenol A; 4,4′-dihydroxydiphenylmethane; 4,4′-thiodiphenol; 4,4′-sulfonyldiphenol; 2,2′-sulfonyldiphenol; 4,4′-dihydroxydiphenyl oxide; 4,4′-dihydroxybenzophenone; 1,1-bis(4-hydroxyphenyl)-1-phenylethane; 4,4′-bis (4(4-hydroxyphenoxy)-phenylsulfone)diphenyl ether; 4,4′-dihydroxydiphenyl disulfide; 3,3′,3,5′-tetrachloro-4,4′-isopropylidenediphenol; 3,3′,3,5′-tetrabromo-4,4′-isopropylidenediphenol; 3,3′-dimethoxy-4,4′-isopropylidenediphenol; 4,4′-dihydroxybiphenyl; 4,4′-dihydroxy-alpha-methylstilbene; 4,4′-dihydroxybenzanilide; bis(4-hydroxyphenyl)terephthalate; N,N′-bis(4-hydroxyphenyl)terephthalamide; bis(4′-hydroxybiphenyl)terephthalate; 4,4′-dihydroxyphenylbenzoate; bis(4′-hydroxyphenyl)-1,4-benzenediamine; 1,1′-bis(4-hydroxyphenyl)cyclohexane; phloroglucinol; pyrogallol; 2,2′,5,5′-tetrahydroxydiphenylsulfone; tris(hydroxyphenyl)methane; dicyclopentadiene diphenol; tricyclopentadienediphenol; terephthalic acid; isophthalic acid; 4,4′-benzanilidedicarboxylic acid; 4,4′-phenylbenzoatedicarboxylic acid; 4,4′-stilbenedicarboxylic acid; adipic acid; and any combination thereof.

Preparation of the aforementioned advancement reaction products can be performed using known methods, which usually includes combining an epoxy resin with one or more suitable compounds having an average of more than one reactive hydrogen atom per molecule. The reactive hydrogen atom is the hydrogen atom which is reactive with an epoxide group in the epoxy resin. The ratio of the compound having more than one reactive hydrogen atom per molecule to the epoxy resin is generally from about 0.01:1 to about 0.95:1, preferably from about 0.05:1 to about 0.8:1, and more preferably from about 0.10:1 to about 0.5:1 equivalents of the reactive hydrogen atom per equivalent of the epoxide group in the epoxy resin.

Examples of the compounds having an average of more than one reactive hydrogen atom per molecule include dihydroxyaromatic, dithiol, disulfonamide or dicarboxylic acid compounds or compounds containing one primary amine or amide group, two secondary amine groups, one secondary amine group and one phenolic hydroxy group, one secondary amine group and one carboxylic acid group, or one phenolic hydroxy group and one carboxylic acid group, and any combination thereof.

The advancement reaction may be conducted, in the presence or absence of a solvent, with the application of heat and mixing.

The advancement reaction may be conducted at atmospheric, superatmospheric or subatmospheric pressures and at temperatures of from about 20° C. to about 260° C., preferably, from about 80° C. to about 240° C., and more preferably from about 100° C. to about 200° C.

The time required to complete the advancement reaction depends upon the factors such as the temperature employed, the chemical structure of the compound having more than one reactive hydrogen atom per molecule employed, and the chemical structure of the epoxy resin employed. Higher temperature may require shorter reaction time whereas lower temperature require longer period of the reaction time.

In general, the time for completion of the advancement reaction may range from about 5 minutes to about 24 hours, preferably from about 30 minutes to about 8 hours, and more preferably from about 30 minutes to about 4 hours.

A catalyst may also be added in the advancement reaction. Examples of the catalyst may include phosphines, quaternary ammonium compounds, phosphonium compounds and tertiary amines. The catalyst may be employed in quantities from about 0.01 percent to about 3 percent, preferably from about 0.03 percent to about 1.5 percent, and more preferably from about 0.05 percent to about 1.5 percent by weight based upon the total weight of the epoxy resin.

Other details concerning an advancement reaction useful in the present invention are given in U.S. Pat. No. 5,736,620 and Handbook of Epoxy Resins by Henry Lee and Kris Neville, incorporated herein by reference.

The adduct of the present invention is a reaction product of the glycidyl ethers and esters (A), the compound (B) and, optionally, the epoxy resin compound (C).

Adducts formed by the reactions of (A) with (B) or (A) and (B) with (C) may be essentially free of epoxide groups, and contain active hydrogen atoms reactive with an epoxide group.

Adducts formed by the reaction of (A) with (B) or (A) and (B) with (C) may be used to form thermosettable (curable) mixtures. For example, these adducts may be combined with one or more epoxy resins to form a thermosettable mixture. Thermosettable mixtures described herein may also include curing agents and/or catalysts for curing epoxy resins.

The thermosettable (curable) mixtures may also be cured to form a cured product. For example, cured products may include electrical or structural laminates or composites, filament windings, moldings, castings, encapsulation, and the like.

According to the present invention, a sufficient amount of the glycidyl ether amide and glycidyl ester amides (A) and the epoxy resin compound (C), if used, and an excess amount of the compound (B) are provided to the reaction to form the adduct of the present invention. At the end of the reaction, essentially all of the epoxide groups in the glycidyl ether and ester amides (A) and the epoxy resin (C), when used, are reacted with the reactive hydrogen atoms in the compound (B). The unreacted compound (B) may be removed at the end of the reaction or may remain as a part of the adduct product.

In general, the ratio of the compound (B) and the glycidyl ether and ester amides (A) is from about 2:1 to about 100:1, preferably from about 3:1 to about 60:1, and more preferably from about 4:1 to about 40:1 equivalents of the reactive hydrogen atom in the compound (B) per equivalent of epoxide group in the glycidyl ethers and esters (A) and resin compound (C), if used.

A catalyst may be employed to prepare adducts described herein. Examples of the catalyst include phosphines, quaternary ammonium compounds, phosphonium compounds and tertiary amines, and any mixture thereof. The amount of catalyst used, if any, depends upon the particular reactants used for preparing the adduct and the type of catalyst employed. In general, the catalyst may be used in an amount of from about 0.01 percent to about 1.5 percent, and preferably from about 0.03 percent to about 0.75 percent by weight based on the total weight of the adduct.

One or more solvents may be present in the reaction to form embodiments of adducts described herein. The presence of the solvent or solvents can improve the solubility of the reactants or, if the reactant is in a solid form, dissolve the solid reactant for easy mixing with other reactants. The presence of the solvent may also dilute the concentration of the reactants in order to moderate the adduct forming reaction such as to control heat generated from the adduct forming reaction or to lower the effective concentration of a reactant which can in turn influence the structure of the adduct product, for example, produce an adduct with less oligomeric component.

The solvent may be any solvent which is substantially inert to the process of forming the adduct including inert to the reactants, the intermediate products if any, and the final products. Examples of the suitable solvents useful in the present invention include aliphatic, cycloaliphatic and aromatic hydrocarbons, halogenated aliphatic and cycloaliphatic hydrocarbons, aliphatic ethers, aliphatic nitriles, cyclic ethers, glycol ethers, esters, ketones, amides, sulfoxides, and any combination thereof.

Preferred examples of the solvents include pentane, hexane, octane, cyclohexane, methylcyclohexane, toluene, xylene, methylethylketone, methylisobutylketone, cyclohexanone, cyclopentanone, N,N-dimethylformamide, dimethylsulfoxide, diethyl ether, tetrahydrofuran, 1,4-dioxane, dichloromethane, chloroform, ethylene dichloride, methyl chloroform, ethylene glycol dimethyl ether, N,N-dimethylacetamide, acetonitrile, and any combination thereof.

The solvent may be removed at the completion of the reaction using conventional means, such as, for example, vacuum distillation. Alternatively, the solvent may also be left in the adduct product to provide a solvent borne adduct which may be used later, for example, in the preparation of coating or film.

The reaction conditions for forming the adducts of the present invention vary depending upon factors such as types and amounts of reactants employed, type and amount of catalyst used, if any, type and amount of solvent used, if any, and modes of addition of the reactants employed.

For example, the reaction for forming the adducts of the present invention may be conducted at atmospheric pressure (for example, 760 mm Hg), superatmospheric or subatmospheric pressures and at temperatures from about 0° C. to about 260° C., preferably from about 20° C. to about 200° C., and more preferably from about 35° C. to about 160° C.

The time required to complete the reaction for forming the adducts of the present invention depends not only upon the aforementioned factors, but also upon the temperature employed. Higher temperature requires a shorter period of time, whereas lower temperature requires a longer period of time. In general, the time to complete the reaction of forming the adduct of the present invention is preferred to be from about 5 minutes to about one week, more preferably from about 30 minutes to about 72 hours, and most preferably from about 60 minutes to 48 hours.

The time and temperature of the reaction for forming the adduct of the present invention may have significant impact on the distribution of components in the formation of the adduct of the present invention. For example, with higher reaction temperature, shorter reaction time, and with the compound (B) comprises the material having only two reactive hydrogen atoms per molecule, the reaction favors the formation of the adduct with more oligomeric components. The reaction favors the formation of the adduct with more branched or crosslinked components when the compound (B) comprises the material having more than two reactive hydrogen atoms per molecule.

The glycidyl ethers and esters (A) may be directly mixed together with the compound (B), added to the compound (B) in incremental steps, or added to the compound (B) continuously. In addition, one of more solvents may be added to the glycidyl ethers and esters (A) and/or the compound (B) before mixing the glycidyl ethers and esters (A) and the compound (B).

If incremental addition of the glycidyl ethers and esters (A) is used, all or a part of an added increment may be allowed to react prior to addition of the next increment. The incremental addition of the glycidyl ethers and esters (A) reacted with excess amount of the compound (B) generally favors the formation of an adduct composed of material wherein the terminal epoxide groups have been reacted in a ring opening reaction with compound (B), and lesser amounts or free of oligomeric components.

Various post treatments may be applied to the process of preparing the adduct of the present invention in order to modify: (1) the distribution of components of the adduct, (2) the reactivity of the adduct, or (3) the physical properties of the adduct.

For example, for an adduct prepared from a reaction between the glycidyl ethers and esters derived from an alkanolamide (as component (A)) and cyclohexylamine (as the compound (B)), when a large stoichiometric excess amount of the primary amine groups derived from the cyclohexylamine reacts with the epoxide groups derived from the diglycidyl ethers or esters derived from an alkanolamide, the reaction may lead to the formation of the adduct with a low content of oligomeric component. The finished product may also comprise, as a part of the adduct, a high concentration of cyclohexylamine as the unreacted compound (B). Accordingly, post treatment of the adduct, such as vacuum distillation, may be employed to strip out the unreacted compound (B).

Other post treatment methods used to modify the distribution of the adduct components may also be employed, such as, for example, recrystallization, chromatographic separation, extraction, zone refining, crystal refining, wiped film distillation, simple distillation, preferential chemical derivatization and removal of one or more components of the adduct, and any combination thereof.

According to embodiments disclosed herein, the reaction of the glycidyl ethers and esters (A) and the compound (B) to form the adduct of the present invention involves a ring opening reaction. During the ring opening reaction, the epoxide groups in the glycidyl ethers and esters (A) reacts with the reactive hydrogen atoms in the compound (B) to give characteristic 2-hydroxylpropyl functionalities as linkages between residual structures of the glycidyl ethers and esters (A) and residual structures of the compound (B).

An example of this type of structure in the adducts of the present invention is shown by the reaction of a glycidyl ethers and esters derived from an alkanolamide (A) and cyclohexylamine (as the compound (B)) (only a partial structure is shown below as Formula XVII).

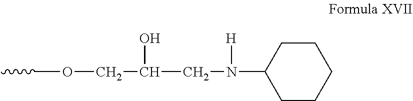

The compound (B) may include compounds having dual functional groups, such as (f) sulfonamides, (g) aminophenols, (h) aminocarboxylic acids, (i) phenolic hydroxyl containing carboxylic acids, and (j) sulfanilamides. These compounds may be utilized to provide an adduct with different functional groups of different reactivity for curing an epoxy resin.

An example of this type of adduct is a reaction product of an aminophenol compound, p-N-methylaminomethylphenol (as the compound (B)), and the glycidyl ethers and esters (A). The reaction provides the adduct with additional phenolic hydroxyl terminated groups. The reaction is performed under mild conditions including (a) with no catalyst, (b) at low temperature (for example, from about 25° C. to about 50° C.), (c) for long reaction time, (d) using incremental or slow continuous addition of the glycidyl ethers and esters (A) to a large stoichiometric excess of the compound (B), and (e) both the glycidyl ether amides and glycidyl ester amides (A) and the compound (B) are in solvent. The following adduct structure shows the adduct comprising phenolic hydroxyl terminated groups (only a partial structure is shown below as Formula XVIII):

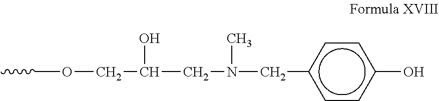

A catalysis reaction favoring one functional group over another with the epoxide group may also be employed. For example, when a compound (B) comprising at least two different functional groups each bearing at least one reactive hydrogen atom is used to form embodiments of an adduct described herein, a catalyst which favors a reaction of reactive hydrogen atom(s) of one type of functional group with an epoxide group over a reaction of reactive hydrogen atom(s) of the other type of functional group with an epoxide group may be employed.

The adduct may also comprise oligomeric components derived from a reaction of epoxide groups from at least two separate epoxy resin molecules with each respective epoxy resin having one of the epoxide groups already reacted with the reactive hydrogen atoms in the compound (B).

An example of this type of adduct is a reaction product of glycidyl ethers and esters derived from an alkanolamide and a cyclohexylamine. The following adduct structure shows that in the oligomeric component derived from at least two epoxide groups from two separate polyglycidyl ethers derived from an alkanolamide each with one of the epoxide groups already reacted with cyclohexylamine, wherein n has a value of one or more, as illustrated in Formula XIX as follows (only a partial structure is shown):

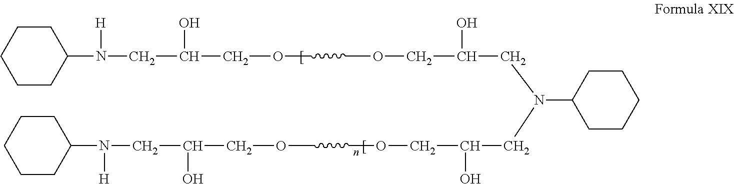

The adduct may also comprise at least one branched or crosslinked structure derived from the following reactions:

(1) a reaction between an epoxide group and the hydroxyl group of a 2-hydroxypropyl linkage contained in the adduct;

(2) a reaction between three separate epoxy resins with three reactive hydrogen atoms from the compound (B).

An example of the above reaction (1) is a reaction of a hydroxyl group in an adduct of the diglycidyl ether of an alkanolamide and cyclohexylamine with an epoxide group from a second diglycidyl ether of an alkanolamide which has already been adducted with cyclohexylamine at one of the epoxide groups, and is illustrated by Formula XX as follows (only a partial structure is shown):

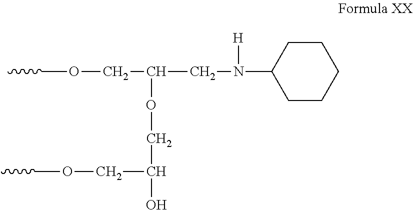

An example of the above reaction (2) is a reaction of an amino hydrogen of the adduct of diethylenetriamine and the diglycidyl ether of an alkanolamide wherein an epoxide group from a second diglycidyl ether of an alkanolamide which has already reacted with another amino hydrogen in the diethylenetriamine moiety, and is illustrated by Formula XXI as follows (only a partial structure is shown):

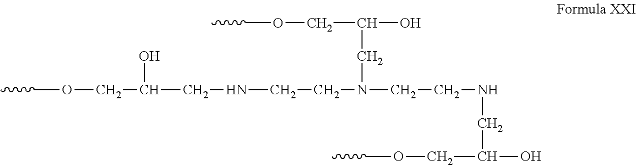

In addition, some minor structures may also be present in embodiments of the adducts described herein, such as, for example, 1,2-glycol group derived from hydrolysis of the epoxide group, or halomethyl groups derived from the addition of epihalohydrin to the hydroxyl group of an intermediate halohydrin molecule. Other minor structures may be formed via a reaction of a backbone hydroxyl group in the adduct of the diglycidyl ether. For example, a reaction of the secondary hydroxyl group with a carboxylic acid group present in some of the compound (B), results in the formation of a backbone ester linkage in the adduct.

The adduct may also contain unreacted compound (B). Thus, in the aforementioned example of the reaction of the glycidyl ether and cyclohexylamine, if the stoichiometric excess of cyclohexylamine employed is not removed, it thus becomes a part of the adduct product.

The curable epoxy resin composition of the present disclosure comprises (a) an adduct of the present invention as described above, and (b) an epoxy resin compound (D); wherein the adduct comprises at least one reaction product of a glycidyl ether or ester (A) and a compound (B), each as described above. The epoxy resin compound (D) comprises one or more epoxy resins. The curable epoxy resin composition, when cured, provides a cured epoxy resin comprising the glycidyl ether or ester derived from alkanolamides.

The term “curable” (also referred to as “thermosettable”) means that the composition is capable of being subjected to conditions which will render the composition to a cured or thermoset state or condition.

The term “cured” or “thermoset” is defined by L. R. Whittington in Whittington's Dictionary of Plastics (1968) on page 239 as follows: “Resin or plastics compounds which in their final state as finished articles are substantially infusible and insoluble. Thermosetting resins are often liquid at some stage in their manufacture or processing, which are cured by heat, catalysis, or some other chemical means. After being fully cured, thermosets cannot be resoftened by heat. Some plastics which are normally thermoplastic can be made thermosetting by means of crosslinking with other materials.”

One embodiment of the present invention is a curable epoxy resin composition of the present disclosure prepared by mixing the adduct of the present invention and resin compound (D) in sufficient amounts of each component to provide a curable epoxy resin composition. The epoxy resin which may be used as the resin compound (D) for the curable epoxy resin composition of the present disclosure may be any epoxide-containing compound which has an average of more than one epoxide group per molecule. Examples of the epoxy resin include those epoxy resins which are suitable for the epoxy resin compound (C) and the epoxy resin material (A) described above.

Generally, the ratio of the adduct of the present disclosure and the resin compound (D) is from about 0.60:1 to about 1.50:1, and preferably from about 0.95:1 to about 1.05:1 equivalents of reactive hydrogen atom present in the adduct per equivalent of epoxide group in the resin compound (D) at the conditions employed for curing.

A preferred curable epoxy resin composition of the present invention comprises an adduct of the present invention and the resin compound (D), wherein the resin compound (D) comprises one or more of epoxy resins selected from the glycidyl ether (A) described above, for example the one or more epoxy resins comprises a glycidyl ether derived from an alkanolamide functionalized fatty acid ester or fatty acid triglyceride.

Another preferred curable epoxy resin composition of the present invention comprises the adduct of the present disclosure and the resin compound (D), wherein the resin compound (D) comprises one or more epoxy resins, and the adduct comprises at least one reaction product of a glycidyl ether (A) and a compound (B). The compound (B) comprises an aliphatic or cycloaliphatic diamine, an aliphatic or cycloaliphatic polyamine, or any combination thereof.

Another embodiment of the curable compositions described herein may include one or more of the adducts, described above, and a blend of one or more of the glycidyl ethers and esters (A) with one or more epoxy resins (D). For example, curable compositions may include one or more adducts of an aliphatic or cycloaliphatic diamine, an aliphatic or cycloaliphatic polyamine, or any combination thereof, and a blend of one or more of the glycidyl ethers (A) with one or more epoxy resins of an aromatic bisphenol, such as, for example, bisphenol A. The resulting curable compositions may result in a thermoset matrix containing aromatic groups in an amount sufficient to enhance mechanical properties while maintaining a desired level of weatherability (resistance to ultraviolet radiation, moisture, etc).

The process of curing the curable epoxy resin composition of the present invention may be conducted at a predetermined curing pressure, at a predetermined temperature and for a predetermined period of time. In general, the curing pressure may be at atmospheric pressure (for example, 760 mm Hg), superatmospheric or subatmospheric pressures; and the temperature may be at a temperature from about 0° C. to about 300° C., preferably from about 25° C. to about 250° C., and more preferably from about 50° C. to about 200° C.

The time required to complete the curing may depend upon the temperature employed. Higher temperatures generally require a shorter period of time whereas lower temperatures generally require longer periods of time. In general, the required time for completion of the curing is from about 1 minute to about 48 hours, preferably from about 15 minutes to about 24 hours, and more preferably from about 30 minutes to about 12 hours.

It is also operable to partially cure (B-stage) the curable epoxy resin composition of the present invention and then complete the curing process at a later time.

The curable epoxy resin composition of the present invention may also comprise a curing agent and/or a curing catalyst in an amount which will effectively cure the curable epoxy resin composition, with the understanding that the amounts will depend upon the specific adduct and epoxy resins employed.

Examples of the curing agent and/or catalyst include aliphatic, cycloaliphatic, polycycloaliphatic or aromatic primary monoamines, aliphatic, cycloaliphatic, polycycloaliphatic or aromatic primary and secondary polyamines, carboxylic acids and anhydrides thereof, aromatic hydroxyl containing compounds, imidazoles, guanidines, urea-aldehyde resins, melamine-aldehyde resins, alkoxylated urea-aldehyde resins, alkoxylated melamine-aldehyde resins, amidoamines, epoxy resin adducts, and any combination thereof.

Particularly preferred examples of the curing agent include, for example, methylenedianiline, 4,4′-diaminostilbene, 4,4′-diamino-alpha-methylstilbene, 4,4′-diaminobenzanilide, dicyandiamide, ethylenediamine, diethylenetriamine, triethylenetetramine, tetraethylenepentamine, urea-formaldehyde resins, melamine-formaldehyde resins, methylolated urea-formaldehyde resins, methylolated melamine-formaldehyde resins, phenol-formaldehyde novolac resins, cresol-formaldehyde novolac resins, sulfanilamide, diaminodiphenylsulfone, diethyltoluenediamine, t-butyltoluenediamine, bis-4-aminocyclohexylamine, isophoronediamine, diaminocyclohexane, hexamethylenediamine, piperazine, 1-(2-aminoethyl)piperazine, 2,5-dimethyl-2,5-hexanediamine, 1,12-dodecanediamine, tris-3-aminopropylamine, and any combination thereof.

Particularly preferred examples of the curing catalyst include boron trifluoride, boron trifluoride etherate, aluminum chloride, ferric chloride, zinc chloride, silicon tetrachloride, stannic chloride, titanium tetrachloride, antimony trichloride, boron trifluoride monoethanolamine complex, boron trifluoride triethanolamine complex, boron trifluoride piperidine complex, pyridine-borane complex, diethanolamine borate, zinc fluoroborate, metallic acylates such as stannous octoate or zinc octoate, and any combination thereof.

The curing catalyst may be employed in an amount which will effectively cure the curable epoxy resin composition. The amount of the curing catalyst will also depend upon the particular adduct, epoxy resin and curing agent, if any, employed in the curable epoxy resin composition.

Generally, the curing catalyst may be used in an amount of from about 0.001 percent to about 2 percent by weight of the total curable epoxy resin composition. In addition, one or more of the curing catalysts may be employed to accelerate or otherwise modify the curing process of the curable epoxy resin composition.

The curing agent may be employed in conjunction with the adduct to cure the curable epoxy resin composition. The amounts of combined curing agent and adduct are from about 0.60:1 to about 1.50:1, and preferably from about 0.95:1 to about 1.05:1 equivalents of reactive hydrogen atom collectively in the curing agent and the adduct.

The curable epoxy resin composition may also be blended with at least one additive including, for example, a cure accelerator, a solvent or diluent, a modifier such as a flow modifier and/or a thickener, a reinforcing agent, a filler, a pigment, a dye, a mold release agent, a wetting agent, a stabilizer, a fire retardant agent, a surfactant, or any combination thereof.

The additive may be blended with the adduct or with the resin compound (D) or with both the adduct and the resin compound (D) prior to use for the preparation of the curable epoxy resin composition of the present invention.

These additives may be added in functionally equivalent amounts, for example, the pigment and/or dye may be added in quantities which will provide the composition with the desired color. In general, the amount of the additives may be from about zero percent to about 20 percent, preferably from about 0.5 percent to about 5 percent, and more preferably from about 0.5 percent to about 3 percent by weight based upon the total weight of the curable epoxy resin composition.

The cure accelerator which can be employed herein includes, for example, mono-, di-, tri- and tetraphenols; chlorinated phenols; aliphatic or cycloaliphatic mono or dicarboxylic acids; aromatic carboxylic acids; hydroxybenzoic acids; halogenated salicylic acids; boric acid; aromatic sulfonic acids; imidazoles; tertiary amines; aminoalcohols; aminopyridines; aminophenols, mercaptophenols; and any mixture thereof.

Particularly suitable cure accelerators include 2,4-dimethylphenol, 2,6-dimethylphenol, 4-methylphenol, 4-tertiary-butylphenol, 2-chlorophenol, 4-chlorophenol, 2,4-dichlorophenol, 4-nitrophenol, 1,2-dihydroxybenzene, 1,3-dihydroxybenzene, 2,2′-dihydroxybiphenyl, 4,4′-isopropylidenediphenol, valeric acid, oxalic acid, benzoic acid, 2,4-dichlorobenzoic acid, 5-chlorosalicylic acid, salicylic acid, p-toluenesulfonic acid, benzenesulfonic acid, hydroxybenzoic acid, 4-ethyl-2-methylimidazole, 1-methylimidazole, triethylamine, tributylamine, N,N-diethylethanolamine, N,N-dimethylbenzylamine, 2,4,6-tris(dimethylamino)phenol, 4-dimethylaminopyridine, 4-aminophenol, 2-aminophenol, 4-mercaptophenol, and any combination thereof.

Examples of the solvent or diluent which can be employed herein include, for example, aliphatic and aromatic hydrocarbons, halogenated aliphatic hydrocarbons, aliphatic ethers, aliphatic nitriles, cyclic ethers, glycol ethers, esters, ketones, amides, sulfoxides, and any combination thereof.

Particularly suitable solvents include pentane, hexane, octane, toluene, xylene, methylethylketone, methylisobutylketone, N,N-dimethylformamide, dimethylsulfoxide, diethyl ether, tetrahydrofuran, 1,4-dioxane, dichloromethane, chloroform, ethylene dichloride, methyl chloroform, ethylene glycol dimethyl ether, diethylene glycol methyl ether, dipropylene glycol methyl ether, N-methylpyrrolidinone, N,N-dimethylacetamide, acetonitrile, sulfolane, and any combination thereof.

The modifier such as the thickener and the flow modifier may be employed in amounts of from zero percent to about 10 percent, preferably, from about 0.5 percent to about 6 percent, and more preferably from about 0.5 percent to about 4 percent by weight based upon the total weight of the curable epoxy resin blend composition.

The reinforcing material which may be employed herein includes natural and synthetic fibers in the form of woven fabric, mat, monofilament, multifilament, unidirectional fiber, roving, random fiber or filament, inorganic filler or whisker, or hollow sphere. Other suitable reinforcing material includes glass, ceramics, nylon, rayon, cotton, aramid, graphite, polyalkylene terephthalates, polyethylene, polypropylene, polyesters, and any combination thereof.

The filler which may be employed herein includes, for example, inorganic oxide, ceramic microsphere, plastic microsphere, glass microsphere, inorganic whisker, calcium carbonate, and any combination thereof.

The filler may be employed in an amount from about zero percent to about 95 percent, preferably from about 10 percent to about 80 percent, and more preferably from about 40 percent to about 60 percent by weight based upon the total weight of the curable epoxy resin composition.

The adduct of the present invention is useful as a curing agent for producing a cured epoxy resin, including in specific cases, production of fully cycloaliphatic/aliphatic cured epoxy resin (with no aromatic rings).

The adduct may be used as, for example, (a) a reactant for thermoset polyurethanes and polyureaurethanes and as (b) an initiator for diols/polyols useful in preparation of thermoset polyurethanes and polyureaurethanes.

The adduct may also be employed in, for example, coatings, especially protective coatings with excellent solvent resistant, moisture resistant, abrasion resistant, and weatherable properties. Other applications of the adduct of the present invention may include, for example, preparation of electrical or structural laminate or composite, filament windings, moldings, castings, encapsulation, and the like.

### EXAMPLES

The following standard analytical equipment and methods are used in the Examples and Comparative Experiments:

### Percent Epoxide/Epoxide Equivalent Weight (EEW) Analysis

A standard titration method was used to determine percent epoxide in the various epoxy resins. A sample was weighed (ranging from about 0.1-0.2 g) and dissolved in dichloromethane (15 mL). Tetraethylammonium bromide solution in acetic acid (15 mL) was added to the sample. The resultant solution was treated with 3 drops of crystal violet solution (0.1% w/v in acetic acid) and was titrated with 0.1N perchloric acid in acetic acid on a Metrohm 665 Dosimat titrator (Brinkmann). Titration of a blank sample comprising dichloromethane

(15 mL) and tetraethylammonium bromide solution in acetic acid (15 mL) provided correction for solvent background. General methods for this titration are found in the scientific literature, for example, Jay, R. R., “Direct Titration of Epoxy Compounds and Aziridines”, Analytical Chemistry, 36, 3, 667-668 (March, 1964).

The following examples and comparative experiments further illustrate the present invention in detail but are not to be construed to limit the scope thereof.

### Example 1

### A. Synthesis of an Epoxy Resin of a Methyl Hydroxymethylstearate Amide Triol

A one liter, three neck, glass bottom reactor was charged under nitrogen with epichlorohydrin (226.2 grams, 2.444 moles), sodium hydroxide (pellets, anhydrous, reagent grade, ≧98%) (53.8 grams, 1.344 moles), and sodium sulfate (granular, anhydrous) (104.1 grams, 0.733 mole). The reactor was additionally equipped with a condenser (maintained at −3° C.), a thermometer, a Claisen adaptor, an overhead nitrogen inlet (1 LPM N₂ used), and a stirrer assembly (TEFLON paddle, glass shaft, variable speed motor). Methyl hydroxymethylstearate amide triol (82.97 grams, 0.60 hydroxyl equivalent) was added to a side arm vented addition funnel, and then attached to the reactor. The methyl hydroxylmethylstearate amide triol used was a vacuum distilled product consisting predominately of the methyl hydroxylmethylstearate amide triols (138.288 hydroxyl equivalent weight). High pressure liquid chromatographic (HPLC) analysis of this amide triol demonstrated the presence of 41.4 area % and 47.5 area % of the pair of amide triols, with the balance consisting of 14 minor components ranging from 0.34 area % to 2.41 area %. All glassware used for the epoxidation reaction was pre-dried in an oven for ≧48 hours at 150° C. Stirring and heating commenced to give a 25° C. slurry of sodium hydroxide and sodium sulfate in epichlorohydrin. After 15 minutes the stirred slurry equilibrated at 40° C. and dropwise addition of the methyl hydroxymethylstearate amide triol to the stirred slurry in the reactor commenced. The reaction temperatures was maintained at 40° C. throughout the 205 minute dropwise addition of the methyl hydroxymethylstearate amide tril. At the end of the addition, the product was a viscous, light tan colored slurry. Additional epichlorohydrin (75.0 grams) was added to reduce the viscosity. The reaction product was held at 40° C. for the next 15.25 hours then sampled for HPLC analysis. Full conversion of the amide triols was achieved using a single product peak (the two isomers do not resolve under the conditions of the HPLC analysis) for the diglycidyl ether comprising 77.6 area % accompanied by a pair of barely resolved product peaks comprising 6.86 area % and 8.30 area % proposed to be the isomeric monoglycidyl ethers, with the balance consisting of 7 minor product peaks ranging from

0.42 area % to 1.69 area %. Heating of the thin, tan colored slurry ceased, followed by addition of methylisobutylketone (400 milliliters), removal of the heating mantle from the reactor, and cooling of the reactor exterior to 25° C. with a fan. The product slurry was equally divided into 4 high density polyethylene bottles which were sealed and centrifuged at 3000 rpm from one hour. The top layer of clear liquid was decanted through a 1.5 inch pad of diatomaceous earth supported on a 600 milliliter coarse fritted glass funnel using vacuum. The bed of diatomaceous earth was prepared by packing 0.5 inch of CELITE 545, followed by 0.5 inch of CELITE standard Super-Cel, then 0.5 inch of CELITE 545. The filtrate was a transparent, light amber colored liquid. The solids remaining in the bottles were equally diluted using fresh methylisobutylketone (each bottle was filled to 250 grams total weight), then placed on the mechanical shaker for one hour, followed by centrifuging and decantation, as previously described. The combined filtrate was a transparent, light amber colored solution. Rotary evaporation of the filtrate using a maximum oil bath temperature of 70° C. to remove the bulk of the volatiles followed by holding one hour using a maximum oil bath temperature of 140° C. provided 111.8 grams of transparent, light amber yellow colored liquid. GC analysis revealed that all light boiling components had been removed. Titration of a pair of aliquots of the product obtained demonstrated an average of 20.93% epoxide (205.1 epoxide equivalent weight). Infrared spectrophotometric analysis of neat thin films of both the amide triol reactant and the triglycidyl ether thereof on a KCl plate confirmed the structure:

(1) Maintenance of the integrity of the amide linkage in the triglycidyl ether at 1645.2 cm⁻¹ for the triglycidyl ether and 1621.8 cm⁻¹ for the amide triol reactant.

(2) Conversion of hydroxyl groups at 3366.1 cm⁻¹ in the amide triol reactant with only a minor hydroxyl absorbance present in the triglycidyl ether at 3451.6 cm⁻¹.

(3) Appearance of a strong aliphatic ether C—O stretch at 1109.5 cm⁻¹ in the triglycidyl ether.

(4) Appearance of epoxide ether C—O stretch at 1253.2 cm⁻¹, 910.6 cm⁻¹ and 848.8 cm⁻¹ in the triglycidyl ether.

(5) Both the amide triol reactant and the triglycidyl ether product possessed minor absorbance (1737.0 cm⁻¹ and 1733.4 cm⁻¹, respectively) which may be indicative of a slight amount of ester functionality.

### B. Preparation and Characterization of an Adduct of Diethylenetriamine and the Epoxy Resin of Methyl Hydroxymethylstearate Amide Triol

A one liter, 3 neck, glass, round bottom, reactor was charged under nitrogen with diethylenetriamine (474.98 grams, 4.605 moles). The reactor was additionally equipped with a condenser (maintained at 0° C.), a thermometer, a Claisen adaptor, an overhead nitrogen inlet (1 LPM N₂ used), and a stirrer assembly (TEFLON paddle, glass shaft, variable speed motor). A portion (37.88 grams, 0.1842 epoxide equivalent) of the epoxy resin of a methyl hydroxymethylstearate amide triol from Part A above was added to a side arm vented addition funnel, and then attached to the reactor. Stirring and heating commenced using a thermostatically controlled heating mantle to give a 75° C. solution. Dropwise addition of the epoxy resin of methyl hydroxymethylstearate amide triol commenced while maintaining the 75° C. reaction temperature. After 162 minutes, dropwise addition was completed. The stirred yellow colored solution was maintained at 75° C. for the next 20.5 hours, followed by rotary evaporation using a maximum oil bath temperature of 114° C. with holding for one hour at 114° C. once the bulk of the excess diethylenetriamine had been removed. A total of 55.61 grams of transparent, yellow colored, liquid adduct was recovered. HPLC analysis of an aliquot of the adduct product revealed that full conversion of the epoxy resin of a methyl hydroxymethylstearate amide triol was achieved giving a single product peak proposed to be the adduct of the isomeric diglycidyl ethers comprising 73.7 area % accompanied by a single peak comprising 23.1 area % proposed to be the adduct of the isomeric monoglycidyl ethers, with the balance consisting of one minor product peak comprising 3.2 area %. A portion of the adduct product was analyzed by gel permeation chromatography (GPC). Waters Ultrahydrogel Linear 120 and 150 columns were used in series along with a differential refractometer detector (Agilent 1100 Differential Refractive Index). The columns were maintained at 40° C. in the chromatograph oven. The eluent used was 50:50 water:isopropanol containing 0.1M sodium chloride and 0.1 M ethylenediamine at a flow rate of 1 mL per min. The injection volume was 100 microliters. The sample was diluted in 50:50 water:isopropanol containing 0.1 M sodium chloride and 0.1 M ethylenediamine glycol to a concentration of 0.24%-0.28%. Calibration was performed using Polymer Laboratories Polyethylene glycol standard set, PEG 10, Lot 16. The following results were obtained, where relative standard deviation (RSD) for Mₙ, M_{w}, M_{w}/Mₙ, Mₚ, and M_{z} and M_{z+1} was less than 6%:

| | |
|---|---|
| Mₙ = 1210 | Mₚ = 1040 |
| M_{w} = 1720 | M_{z} = 4150 |
| M_{w}/Mₙ = 1.42 | M_{z+1} = 11000 |

A portion of the adduct product was added to acetic acid (25 mL) then titrated using perchloric acid in acetic acid (0.1) and crystal violet indicator. Calculation using the data from this titration provided an amine hydrogen equivalent weight of 87.84. Repeats of the titration provided amine hydrogen equivalent weights of 91.07, 89.24, and 89.15. All four values are after correction for titration of an acetic acid (25 mL) blank. Averaging gave an amine hydrogen equivalent weight of 89.33.

### Example 2

### Preparation and Characterization of an Adduct of Isophoronediamine and the Epoxy Resin of Methyl Hydroxymethylstearate Amide Triol

A one liter, 3 neck, glass, round bottom, reactor was charged under nitrogen with isophoronediamine (470.60 grams, 2.7634 moles). The reactor was additionally equipped with a condenser (maintained at 0° C.), a thermometer, a Claisen adaptor, an overhead nitrogen inlet (I LPM N2 used), and a stirrer assembly (TEFLON paddle, glass shaft, variable speed motor). A portion (37.88 grams, 0.1842 epoxide equivalent) of the epoxy resin of a methyl hydroxymethylstearate amide triol from Example 1A was added to a side arm vented addition funnel, then attached to the reactor. Stirring and heating commenced using a thermostatically controller heating mantle to give a 75° C. solution. Dropwise addition of the epoxy resin of methylhydroxymethylstearate amide triol commenced while maintaining the 75° C. reaction temperature. After 177 minutes dropwise addition was completed. The stirred yellow colored solution was maintained at 75° C. for the next 18.25 hr followed by rotary evaporation using a maximum oil bath temperature of 114° C. with holding for one hour at 114° C. once the bulk of the excess isophoronediamine had been removed. A total of 71.45 grams of transparent, yellow colored, liquid adduct was recovered. HPLC analysis of an aliquot of the adduct product revealed that full conversion of the epoxy resin of a methylhydroxymethylstearate amide triol was achieved giving major product peaks comprising 62.1 area %, 14.3 area %, and 10.2 area % with the balance consisting of three minor product peak comprising 3.9 area %, 3.9 area %, and 5.6 area %. A portion of the adduct product was analyzed by gel permeation chromatography (GPC). Waters Ultrahydrogel Linear 120 and 150 columns were used in series along with a differential refractometer detector (Agilent 1100 Differential Refractive Index). The columns were maintained at 40° C. in the chromatograph oven. The eluent used was 50:50 water:isopropanol containing 0.1M sodium chloride and 0.1M ethylenediamine at a flow rate of 1 mL per min. The injection volume was 100 microliters. The sample was diluted in 50:50 water:isopropanol containing 0.1 M sodium chloride and 0.1 M ethylenediamine to a concentration of 0.24-0.28%. Calibration was performed using Polymer Laboratories Polyethylene glycol standard set, PEG 10, Lot 16. The following results were obtained, where relative standard deviation (RSD) for Mₙ, M_{w}, M_{w}/Mₙ, Mₚ, and M_{z} and M_{z+1} was less than 6%:

| | |
|---|---|
| Mₙ = 1530 | Mₚ = 1360 |
| M_{w} = 2180 | M_{z} = 4570 |
| M_{w}/Mₙ = 1.43 | M_{z+1} = 10100 |

A portion of the adduct product was added to acetic acid (25 mL) then titrated using perchloric acid in acetic acid (0.1 N) and crystal violet indicator. Calculation using the data from this titration provided an amine hydrogen equivalent weight of 116.69. A repeat of the titration provided an amine hydrogen equivalent weight of 118.00. Both values are after correction for titration of an acetic acid (25 mL) blank. Averaging gave an amine hydrogen equivalent weight of 117.35.

### Example 3

### Preparation and Testing of Coatings Comprising a Bisphenol A Diglycidyl Ether Cured with an Adduct of Diethylenetriamine Adduct and the Epoxy Resin of a Methyl Hydroxymethylstearate Amide Triol

The following were combined and stirred together to obtain a homogeneous mixture: 13.0 grams (0.069 epoxide equivalents) of a commercial grade diglycidyl ether of bisphenol A (D.E.R. 331 epoxy resin, available from The Dow Chemical Company, Midland, Mich., 188.5 epoxide equivalent weight), 6.16 grams (0.069 amine hydrogen equivalents) of the diethylenetriamine adduct from Example 1 Part B (89.22 amine hydrogen equivalent weight), 4.11 grams of benzyl alcohol (premixed with the adduct) and 3 drops of BYK-310 “Surface Modifier” (polyester modified polydimethylsiloxane surface modifier, BYK Chemie USA). A portion of this mixture was applied to 2 unpolished cold roll steel plates using a No. 48 BYK draw down bar and then cured 5 days at room temperature followed by 1 day at 60° C. plus 2 days at 80° C. The cured coatings obtained had an average thickness of 1.89 mils.

The following properties were obtained for these coatings:

Pendulum Hardness (ASTM Method D 4366-95-Method A)=180 seconds

Pencil Hardness (ASTM Method D 3363)=3H

Conical Mandrel Bend (ASTM Method D 522-93a)=No failure

Cross Hatch Adhesion (ASTM Method 3359)=3B Rating (5 to 15% Failure)

Methyl Ethyl Ketone Double Rubs (ASTM Method D 4752-87)=200+

Direct and Reverse Impact Strength (ASTM Method D2794)=104 and 16 in. lbs.

The results are summarized in Table I.

A portion of the mixture comprising the diglycidyl ether of bisphenol A, the diethylenetriamine adduct, benzyl alcohol and BYK-310 was applied to 2 Tru Aluminum unpolished, coil coat white panels (3 inch by 6 inch by 0.038 inch, ACT Laboratories). The coatings obtained on these panels were also cured 5 days at room temperature followed by 1 day at 60° C. plus 2 days at 80° C. After curing, the gloss of the coatings was measured using a gloss meter in accordance with ASTM method D-523. The average gloss (percent light reflectance) at angles of 20°, 60° and 85° for the coated panels were 17.8 percent, 44.4 percent and 76.57 percent, respectively. The panels were then placed in an apparatus described in ASTM Method G-53 and alternately exposed to 4 hours of ultraviolet light at 60° C. and 4 hours of water condensation at 50° C. in a repetitive cycle. The ultraviolet irradiation in this apparatus was from an array of UV-A type lamps operating at a wavelength of 340 nm. To determine the effect of these conditions on the gloss, the panels were briefly removed from the apparatus, approximately, every 100 hours and measurements were made. The results are summarized in Table 3

A portion of the mixture comprising the diglycidyl ether of bisphenol A, the diethylenetriamine adduct, benzyl alcohol and BYK-310 was also applied to two glass drying strips from which the following results were obtained using a B-K Drying Recorder (Paul N. Gardner Company, Inc.):

Set to touch time=1.9 hours

Dust free time=2.25 hours

Dry-through time=2.4 hours

The results are summarized in Table 2.

### Example 4

### Preparation and Testing of Coatings Comprising a Bisphenol A Diglycidyl Ether Cured with an Adduct of Isophoronediamine and the Epoxy Resin of a Methyl Hydroxymethylstearate Amide Triol

The following were combined and stirred together to obtain a homogeneous mixture: 12.0 grams (0.064 epoxide equivalents) of a commercial grade diglycidyl ether of bisphenol A (D.E.R. 331 epoxy resin, available from The Dow Chemical Company, 188.5 epoxide equivalent weight), 7.47 grams (0.064 amine hydrogen equivalent) of the isophoronediamine adduct from Example 2 (117.35 amine hydrogen equivalent weight), 4.98 grams of benzyl alcohol (premixed with the adduct) and 3 drops of BYK-310 “Surface Modifier” (polyester modified polydimethylsiloxane surface modifier, BYK Chemie USA). A portion of this mixture was applied to 2 unpolished cold roll steel plates using a No. 48 BYK draw down bar and then cured 5 days at room temperature followed by 1 day at 60° C. plus 2 days at 80° C. The cured coatings obtained had an average thickness of 1.89 mils. The following properties were obtained for these coatings:

Pendulum Hardness (ASTM Method D 4366-95-Method A)=185 seconds

Pencil Hardness (ASTM Method D 3363)=3H

Conical Mandrel Bend (ASTM Method D 522-93a)=Delaminated

Cross Hatch Adhesion (ASTM Method 3359)=1B Rating (35 to 65% Failure)

Methyl Ethyl Ketone Double Rubs (ASTM Method D 4752-87)=200+

Direct and Reverse Impact Strength (ASTM Method D2794)=64 and 8 in. lbs.

The results are summarized in Table 1.

A portion of the mixture comprising the diglycidyl ether of bisphenol A, the isophorone diamine adduct, benzyl alcohol and BYK-310 was applied to 2 Tru Aluminum unpolished, coil coat white panels (3 inch by 6 inch by 0.038 inch, ACT Laboratories). The coatings obtained on these panels were also cured 5 days at room temperature followed by 1 day at 60° C. plus 2 days at 80° C. After curing, the gloss of the coatings was measured using a gloss meter in accordance with ASTM method D-523. The average gloss (percent light reflectance) at angles of 20°, 60° and 85° for the coated panels were 17.8 percent, 44.4 percent and 76.57 percent, respectively. The panels were then placed in an apparatus described in ASTM Method G-53 and alternately exposed to 4 hours of ultraviolet light at 60° C. and 4 hours of water condensation at 50° C. in a repetitive cycle. The ultraviolet irradiation in this apparatus was from an array of UV-A type lamps operating at a wavelength of 340 nm. To determine the effect of these conditions on the gloss, the panels were briefly removed from the apparatus, approximately, every 100 hours and measurements were made. The results are summarized in Table 3.

A portion of the mixture comprising the diglycidyl ether of bisphenol A, the diethylenetriamine adduct, benzyl alcohol and BYK-310 was also applied to two glass drying strips from which the following results were obtained using a B-K Drying Recorder (Paul N. Gardner Company, Inc.):

Set to touch time=5 hours

Dust free time=6.25 hours

Dry-through time=11.5 hours

The results are summarized in Table 2.

### Comparative Example A

### Preparation and Testing of Coatings Comprising a Bisphenol A Diglycidyl Ether Cured with Diethylenetriamine

The following were combined and stirred together to obtain a homogeneous mixture: 25.0 grams (0.133 epoxide equivalents) of a commercial grade diglycidyl ether of bisphenol A (D.E.R. 331 epoxy resin, available from The Dow Chemical Company, 188.5 epoxide equivalent weight), 2.73 grams (0.133 amine hydrogen equivalent) of the diethylenetriamine (20.6 amine hydrogen equivalent weight), 1.82 grams of benzyl alcohol (premixed with the diethylenetriamine) and 3 drops of BYK-310 “Surface Modifier” (polyester modified polydimethylsiloxane surface modifier, BYK Chemie USA). A portion of this mixture was applied to 2 unpolished cold roll steel plates using a No. 48 BYK draw down bar and then cured 5 days at room temperature followed by 1 day at 60° C. plus 2 days at 80° C. The cured coatings obtained had an average thickness of 1.89 mils. The following properties were obtained for these coatings:

Pendulum Hardness (ASTM Method D 4366-95-Method A)=198 seconds

Pencil Hardness (ASTM Method D 3363)=4H

Conical Mandrel Bend (ASTM Method D 522-93a)=Delaminated

Cross Hatch Adhesion (ASTM Method 3359)=OB Rating (>65% Failure)

Methyl Ethyl Ketone Double Rubs (ASTM Method D 4752-87)=200+

Direct and Reverse Impact Strength (ASTM Method D2794)=52 and <4 in. lbs.

The results are summarized in Table 1.

A portion of the mixture comprising the diglycidyl ether of bisphenol A, diethylenetriamine, benzyl alcohol and BYK-310 was applied to 2 Tru Aluminum unpolished, coil coat white panels (3 inch by 6 inch by 0.038 inch, ACT Laboratories). The coatings obtained on these panels were also cured 5 days at room temperature followed by 1 day at 60° C. plus 2 days at 80° C. After curing, the gloss of the coatings was measured using a gloss meter in accordance with ASTM method D-523. The average gloss (percent light reflectance) at angles of 20°, 60° and 85° for the coated panels were 17.8 percent, 44.4 percent and 76.57 percent, respectively. The panels were then placed in an apparatus described in ASTM Method G-53 and alternately exposed to 4 hours of ultraviolet light at 60° C. and 4 hours of water condensation at 50° C. in a repetitive cycle. The ultraviolet irradiation in this apparatus was from an array of UV-A type lamps operating at a wavelength of 340 nm. To determine the effect of these conditions on the gloss, the panels were briefly removed from the apparatus, approximately, every 100 hours and measurements were made.

The results are summarized in Table 3.

A portion of the mixture comprising the diglycidyl ether of bisphenol A, diethylenetriamine, benzyl alcohol and BYK-310 was also applied to two glass drying strips from which the following results were obtained using a B-K Drying Recorder (Paul N. Gardner Company, Inc.):

Set to touch time=3.6 hours

Dust free time=5.6 hours

Dry-through time=8.1 hours

The results are summarized in Table 2.

### Comparative Experiment B

### Preparation and Testing of Coatings Comprising a Bisphenol A Diglycidyl Ether Cured with Isophoronediamine

The following were combined and stirred together to obtain a homogeneous mixture: 25.00 grams (0.1326 epoxide equivalents) of a commercial diglycidyl ether of bisphenol A (D.E.R. 331 epoxy resin, available from The Dow Chemical Company, 188.5 epoxide equivalent weight), 5.64 grams (0.1325 amine hydrogen equivalents) of isophoronediamine (42.58 amine hydrogen equivalent weight), 3.76 grams of benzyl alcohol (premixed with the isophoronediamine) and 3 drops of BYK-310 “Surface Modifier” (polyester modified polydimethylsiloxane surface modifier, BYK Chemie USA). A portion of this mixture was applied to 3 unpolished, cold roll steel plates using a No. 48 BYK draw down bar and then cured 5 days at room temperature followed by 1 day at 60° C. plus 2 days at 80° C. The cured coatings obtained had an average thickness of 1.84 mils. The following properties were obtained for these coatings:

Pendulum Hardness (ASTM Method D 4366-95-Method A)=188 seconds

Pencil Hardness (ASTM Method D 3363)=2H

Conical Mandrel Bend (ASTM Method D 522-93a)=No Failure

Cross Hatch Adhesion (ASTM Method 3359)=46 Rating (<5% Failure)

Methyl Ethyl Ketone Double Rubs (ASTM Method D 4752-87)=200+

Direct and Reverse Impact Strength (ASTM Method D2794)=160 and 12 in. lbs.

The results are summarized in Table 1.

A portion of the mixture comprising the diglycidyl ether of bisphenol A, the isophoronediamine, benzyl alcohol and BYK-310 was applied to 3 Tru Aluminum unpolished, coil coat white panels (3 inches by 6 inches by 0.038 inch, ACT Laboratories). The coatings obtained on these panels were also cured 5 days at room temperature followed by 1 day at 60° C. plus 2 days at 80° C. After curing, the gloss of the coatings was measured using a gloss meter in accordance with ASTM method D-523. The average gloss (percent light reflectance) at angles of 20° 60° and 85° for the coated panels were 93.3 percent, 100.0 percent and 100.0 percent, respectively. The panels were then placed in an apparatus described in ASTM Method G-53 and alternately exposed to 4 hours of ultraviolet light at 60° C. and 4 hours of water condensation at 50° C. in a repetitive cycle. The ultraviolet irradiation in this apparatus was from an array of UV-A type lamps operating at a wavelength of 340 nm. To determine the effect of these conditions on the gloss, the panels were briefly removed from the apparatus, approximately, every 100 hours and measurements were made. The results are summarized in Table 3.

A portion of the mixture comprising the diglycidyl ether of bisphenol A, isophoronediamine, benzyl alcohol and BYK-310 was also applied to two glass drying strips from which the following results were obtained using a B-K Drying Recorder (Paul N. Gardner Company, Inc.):

Set to touch time=3.9 hours

Dust free time=8.1 hours

Dry-through time=12.75 hours

The results are summarized in Table 2.

### Comparative Example C

### Preparation and Testing of Coatings Comprising a Blend of the Bisphenol A Diglycidyl Ether and the Epoxy Resin of a Methyl Hydroxymethylstearate Amide Triol Cured with Diethylenetriamine

The following were combined and stirred together to obtain a homogeneous mixture: 20.00 grams (0.106 epoxide equivalents) of a commercial grade diglycidyl ether of bisphenol A (D.E.R. 331 epoxy resin, available from The Dow Chemical Company, 188.5 epoxide equivalent weight), 6.00 grams (0.0292 epoxide equivalents) of the epoxy resin of the methyl hydroxymethylstearate amide triol from Example 1 (205.61 epoxide equivalent weight), 2.79 grams (0.1354 amine equivalents) of diethylenetriamine (20.6 amine hydrogen equivalent weight), 6.19 grams of benzyl alcohol and 3 drops of BYK-310 “Surface Modifier” (polyester modified polydimethylsiloxane surface modifier, BYK Chemie USA). This formulation was designed to contain the same amount of the seed oil amide segment and the same amount of benzyl alcohol solvent carrier as in Example 3 (14.4 weight percent and

17.7 weight percent, respectively). A portion of this mixture was applied to 3 unpolished, cold roll steel plates using a No. 48 BYK draw down bar and then cured 5 days at room temperature followed by 1 day at 60° C. plus 2 days at 80° C. The cured coatings obtained had an average thickness of 1.63 mils. The following properties were obtained for these coatings:

Pendulum Hardness (ASTM Method D 4366-95-Method A)=160 seconds

Pencil Hardness (ASTM Method D 3363)=4H

Conical Mandrel Bend (ASTM Method D 522-93a)=39 mm failure

Cross Hatch Adhesion (ASTM Method 3359)=4B (˜5% failure)

Methyl Ethyl Ketone Double Rubs (ASTM Method D 4752-87)=2200 rubs

Direct and Reverse Impact Strength (ASTM Method D2794)=761 <4 in. lbs.

The results are summarized in Table 1.

A portion of the mixture comprising the diglycidyl ether of bisphenol A, the epoxy resin of the methyl hydroxymethylstearate amide triol, diethylenetriamine, benzyl alcohol and BYK-310 was applied to 4 Tru Aluminum unpolished, coil coat white panels (3 inches by 6 inches by 0.038 inch, ACT Laboratories). The coatings obtained on these panels were also cured 5 days at room temperature followed by 1 day at 60° C. plus 2 days at 80° C. After curing, the gloss of the coatings was measured using a gloss meter in accordance with ASTM method D-523. The average gloss (percent light reflectance) at angles of 20° 60° and 85° for the coated panels were 27.9 percent, 55.4 percent and 88.7 percent, respectively. The panels were then placed in an apparatus described in ASTM Method G-53 and alternately exposed to 4 hours of ultraviolet light at 60° C. and 4 hours of water condensation at 50° C. in a repetitive cycle. The ultraviolet irradiation in this apparatus was from an array of UV-A type lamps operating at a wavelength of 340 nm. To determine the effect of these conditions on the gloss, the panels were briefly removed from the apparatus, approximately, every 100 hours and measurements were made. The results are summarized in Table 3.

A portion of the mixture comprising the diglycidyl ether of bisphenol A, the epoxy resin of the methyl hydroxymethylstearate amide triol, diethylenetriamine, benzyl alcohol and BYK-310 was also applied to three glass drying strips from which the following results were obtained using a B-K Drying Recorder (Paul N. Gardner Company, Inc.):

Set to touch time=4.0 hours

Dust free time=4.5 hours

Dry-through time=9.2 hours

The results are summarized in Table 2.

### Example 5

### Preparation and Characterization of an Adduct of bis(Hexamethylene)triamine and the Epoxy Resin of Methyl hydroxymethylstearate Amide Triol

A one liter, 3 neck, glass, round bottom, reactor was charged under nitrogen with bis(hexamethylene)triamine (991.87 grams, 4.605 moles). The reactor was additionally equipped with a condenser (maintained at 0° C.), a thermometer, a Claisen adaptor, an overhead nitrogen inlet (1 LPM N₂ used), and a stirrer assembly (TEFLON paddle, glass shaft, variable speed motor). A portion (37.83 grams, 0.1842 epoxide equivalent) of an epoxy resin of a methyl hydroxymethylstearate amide triol prepared using the method of Example 1A (205.38 epoxide equivalent weight) was added to a side arm vented addition funnel, and then attached to the reactor. Stirring and heating commenced using a thermostatically controlled heating mantle to give a 75° C. solution. Dropwise addition of the epoxy resin of methyl hydroxymethylstearate amide triol commenced while maintaining the 75° C. reaction temperature. After 3.5 hours, dropwise addition was completed. The stirred light amber colored solution was maintained at 75° C. for the next 18.9 hours, followed by rotary evaporation using a condenser temperature of 35° C. and a maximum oil bath temperature of 170° C. with holding for 20 minutes at 170° C. at which time distillation of bis(hexamethylene)triamine had ceased. A total of 97.49 grams of transparent, amber colored, liquid adduct was recovered. HPLC analysis of an aliquot of the adduct product revealed that full conversion of the epoxy resin of a methyl hydroxymethylstearate amide triol was achieved. A portion of the adduct product was added to acetic acid (25 mL) then titrated using perchloric acid in acetic acid (0.1) and crystal violet indicator. Calculation using the data from this titration provided an amine hydrogen equivalent weight of 91.38. A repeat of the titration provided an amine hydrogen equivalent weight of 92.21. Both values are after correction for titration of an acetic acid (25 mL) blank. Averaging gave an amine hydrogen equivalent weight of 91.80.

### Example 6

### Preparation and Characterization of an Adduct of Triethylenetetramine and the Epoxy Resin of Methyl Hydroxymethylstearate Amide Triol

A one liter, 3 neck, glass, round bottom, reactor was charged under nitrogen with triethylenetetramine (673.44 grams, 4.605 moles). The reactor was additionally equipped with a condenser (maintained at 0° C.), a thermometer, a Claisen adaptor, an overhead nitrogen inlet (1 LPM N₂ used), and a stirrer assembly (TEFLON paddle, glass shaft, variable speed motor). A portion (37.83 grams, 0.1842 epoxide equivalent) of the epoxy resin of a methyl hydroxymethylstearate amide triol prepared using the method of Example 1A (205.38 epoxide equivalent weight) was added to a side arm vented addition funnel, and then attached to the reactor. Stirring and heating commenced using a thermostatically controlled heating mantle to give a 75° C. solution. Dropwise addition of the epoxy resin of methyl hydroxymethylstearate amide triol commenced while maintaining the 75° C. reaction temperature. After 2.8 hours, dropwise addition was completed. The stirred light yellow colored solution was maintained at 75° C. for the next 17.2 hours, followed by rotary evaporation using an oil bath temperature of 125° C.-128° C. to remove the bulk of the excess triethylenetetramine followed by increasing the oil bath temperature to 140° C. and holding for 30 minutes at which time distillation of triethylenetetramine had ceased. A total of 73.62 grams of transparent, yellow colored, liquid adduct was recovered. HPLC analysis of an aliquot of the adduct product revealed that full conversion of the epoxy resin of a hydroxymethylstearate amide triol was achieved. A portion of the adduct product was added to acetic acid (25 mL) then titrated using perchloric acid in acetic acid (0.1) and crystal violet indicator. Calculation using the data from this titration provided an amine hydrogen equivalent weight of 75.21. A repeat of the titration provided an amine hydrogen equivalent weight of 82.06. Both values are after correction for titration of an acetic acid (25 mL) blank. Averaging gave an amine hydrogen equivalent weight of 78.63.

### Example 7

### Preparation and Characterization of an Adduct of Triethylenetetramine and the Epoxy Resin of Methyl Hydroxymethylstearate Amide Triol with Incomplete Removal of Excess Triethylenetetramine

A one liter, 3 neck, glass, round bottom, reactor was charged under nitrogen with triethylenetetramine (673.44 grams, 4.605 moles). The reactor was additionally equipped with a condenser (maintained at 0° C.), a thermometer, a Claisen adaptor, an overhead nitrogen inlet (1 LPM N₂ used), and a stirrer assembly (TEFLON paddle, glass shaft, variable speed motor). A portion (37.83 grams, 0.1842 epoxide equivalent) of the epoxy resin of a methyl hydroxymethylstearate amide triol prepared using the method of Example 1 A (205.38 epoxide equivalent weight) was added to a side arm vented addition funnel, and then attached to the reactor. Stirring and heating commenced using a thermostatically controlled heating mantle to give a 75° C. solution. Dropwise addition of the epoxy resin of methyl hydroxymethylstearate amide triol commenced while maintaining the 75° C. reaction temperature. After 2.8 hours, dropwise addition was completed. The stirred light yellow colored solution was maintained at 75° C. for the next 17.3 hours, followed by rotary evaporation using an oil bath temperature of 124° C.-127° C. to remove 563.5 grams of the excess triethylenetetramine into the receiver. A total of 147.07 grams of transparent, yellow colored, liquid adduct was recovered. HPLC analysis of an aliquot of the adduct product revealed that full conversion of the epoxy resin of a hydroxymethylstearate amide triol was achieved. A portion of the adduct product was added to acetic acid (25 mL) then titrated using perchloric acid in acetic acid (0.1) and crystal violet indicator. Calculation using the data from this titration provided an amine hydrogen equivalent weight of 45.51. A repeat of the titration provided an amine hydrogen equivalent weight of 44.68. Both values are after correction for titration of an acetic acid (25 mL) blank. Averaging gave an amine hydrogen equivalent weight of 45.10.

### Example 8

### Preparation and Testing of Coatings Comprising a Bisphenol A Diglycidyl Ether Cured with a bis(Hexamethylene)triamine Adduct of a Seed Oil Glycidyl Ether

The following were combined and stirred together to obtain a homogeneous mixture: 22.00 grams of a commercial diglycidyl ether of bisphenol A (D.E.R.™ 331 epoxy resin by the Dow Chemical Company; 188.5 epoxide equivalent weight; 0.1167 epoxide equivalents), 10.71 grams of the bis(hexamethylene)triamine adduct from Example 5 (91.80 amine hydrogen equivalent weight; 0.1167 amine hydrogen equivalents), and 3 drops of BYK-310. A portion of this mixture was applied to 3 polished, cold roll steel plates (0.8 mm×102 mm×305 mm; coated with Bonderite 1000, iron phosphate, and P-60 chrome) using a No. 48 BYK draw down bar and then cured 2 days at 60° C. The cured coatings obtained had an average thickness of 3.37 mils. The following properties were obtained for these coatings:

Pendulum Hardness (ASTM Method D 4366-95-Method A)=65 seconds

Pencil Hardness (ASTM Method D 3363)=1H

One-Eight Inch, Conical Mandrel Bend (ASTM Method D 522-93a)=100 mm Failure

Cross Hatch Adhesion (ASTM Method 3359)=Rating 5B (No Failure)

Methyl Ethyl Ketone Double Rubs (ASTM Method D 4752-87)=>200

Direct and Reverse Impact Strength (ASTM Method D2794)=84 and 4 inch pounds

A portion of the mixture comprising the diglycidyl ether of bisphenol A, the bis(hexamethylene)triamine adduct, and BYK-310 was applied to 4 Tru Aluminum unpolished, coil coat white panels (3 inches×6 inches×0.038 inch) from ACT Laboratories. The coatings obtained on these panels were also cured 2 days at 60° C. After curing, the gloss of the coatings was measured using a gloss meter according to ASTM method D-523. The average gloss (percent light reflectance) at angles of 20° 60° and 85° for the coated panels were 1.4 percent, 7.0 percent and 51.2 percent, respectively.

A portion of the mixture comprising the diglycidyl ether of bisphenol A, the bis(hexamethylene)triamine adduct, and BYK-310 was also applied to two glass drying strips from which the following results were obtained using a B-K Drying Recorder manufactured by Paul N. Gardner Company, Inc.:

Set to touch time=1.7 hours

Dust free time=2.8 hours

Dry-through time=17.8 hours

### Example 9

### Preparation and Testing of Coatings Comprising a Glycidyl Ether of a 12-Hydroxy Methylstearate Amide Polyol Cured with a bis(Hexamethylene)triamine Adduct of a Seed Oil Glycidyl Ether

The following were combined and stirred together to obtain a homogeneous mixture: 22.00 grams of a glycidyl ether of 12-hydroxy methylstearate amide polyol (197.4 epoxide equivalent weight; 0.1114 epoxide equivalents), 10.23 grams of the bis(hexamethylene)triamine adduct from Example 5 (91.80 amine hydrogen equivalent weight; 0.1114 amine hydrogen equivalents), and 3 drops of BYK-310. A portion of this mixture was applied to 3 polished, cold roll steel plates (0.8×102×305 mm; coated with Bonderite 1000, iron phosphate, and P-60 chrome) using a No. 48 BYK draw down bar and then cured 2 days at 60° C. The cured coatings obtained had an average thickness of 1.02 mils. The following properties were obtained for these coatings:

Pendulum Hardness (ASTM Method D 4366-95-Method A)=37 seconds

Pencil Hardness (ASTM Method D 3363)=<4B

One-Eight Inch, Conical Mandrel Bend (ASTM Method D 522-93a)=No Failure

Cross Hatch Adhesion (ASTM Method 3359)=Rating 3B (5 to 15% Failure)

Methyl Ethyl Ketone Double Rubs (ASTM Method D 4752-87)=>200

Direct and Reverse Impact Strength (ASTM Method D2794)=>160 inch pounds (both cases)

A portion of the mixture comprising the diglycidyl ether of bisphenol A, the bis(hexamethylene)triamine adduct and BYK-310 was applied to 4 Tru Aluminum unpolished, coil coat white panels (3 inches×6 inches×0.038 inch) from ACT Laboratories. The coatings obtained on these panels were also cured 2 days at 60° C. After curing, the gloss of the coatings was measured using a gloss meter according to ASTM method D-523. The average gloss (percent light reflectance) at angles of 20° 60° and 85° for the coated panels were

1.8 percent, 8.7 percent and 25.7 percent, respectively.

A portion of the mixture comprising the diglycidyl ether of bisphenol A, the bis(hexamethylene)triamine adduct, and BYK-310 was also applied to two glass drying strips from which the following results were obtained using a B-K Drying Recorder manufactured by Paul N. Gardner Company, Inc.:

Set to touch time=1.6 hours

Dust free time=2.8 hours

Dry-through time=21.7 hours

### Example 10

### Preparation and Testing of Coatings Comprising a Bisphenol A Diglycidyl Ether Cured with a Triethylenetetramine Adduct of a Seed Oil Glycidyl Ether

The following were combined and stirred together to obtain a homogeneous mixture: 15.00 grams of a commercial diglycidyl ether of bisphenol A (D.E.R.™ 331 epoxy resin by the Dow Chemical Company; 188.5 epoxide equivalent weight; 0.0796 epoxide equivalents), 6.26 grams of the triethylenetetramine adduct from Example 6 (78.66 amine hydrogen equivalent weight; 0.0796 amine hydrogen equivalents), 4.17 grams of benzyl alcohol (premixed with the adduct) and 3 drops of BYK-310. A portion of this mixture was applied to 3 polished, cold roll steel plates (0.8 mm×10 mm 2×305 mm; coated with Bonderite 1000, iron phosphate, and P-60 chrome) using a No. 48 BYK draw down bar and then cured 2 days at 60° C. The cured coatings obtained had an average thickness of 1.66 mils. The following properties were obtained for these coatings:

Pendulum Hardness (ASTM Method D 4366-95-Method A)=166 seconds

Pencil Hardness (ASTM Method D 3363)=1H

One-Eight Inch, Conical Mandrel Bend (ASTM Method D 522-93a)=No Failure

Cross Hatch Adhesion (ASTM Method 3359)=5B Rating (No Failure)

Methyl Ethyl Ketone Double Rubs (ASTM Method D 4752-87)=>200

Direct and Reverse Impact Strength (ASTM Method D2794)=148 and 80 inch pounds

A portion of the mixture comprising the diglycidyl ether of bisphenol A, the triethylenetetramine adduct, benzyl alcohol and BYK-310 was also applied to two glass drying strips from which the following results were obtained using a B-K Drying Recorder manufactured by Paul N. Gardner Company, Inc.:

Set to touch time=1.6 hours

Dust free time=1.9 hours

Dry-through time=4.7 hours

### Example 11

### Preparation and Testing of Coatings Comprising a Bisphenol A Diglycidyl Ether Cured with an Triethylenetetramine Adduct of a Seed Oil Glycidyl Ether

The following were combined and stirred together to obtain a homogeneous mixture: 25.00 grams of a commercial diglycidyl ether of bisphenol A (D.E.R.™ 331 epoxy resin by The Dow Chemical Company; 188.5 epoxide equivalent weight; 0.1326 epoxide equivalents), 5.98 grams of the triethylenetetramine adduct from Example 7 (45.095 amine hydrogen equivalent weight; 0.1326 amine hydrogen equivalents), and 3 drops of BYK-310. A portion of this mixture was applied to 3 polished, cold roll steel plates (0.8 mm×102 mm×305 mm; coated with Bonderite 1000, iron phosphate, and P-60 chrome) using a No. 48 BYK draw down bar and then cured 2 days at 60° C. The cured coatings obtained had an average thickness of 2.53 mils. The following properties were obtained for these coatings:

Pendulum Hardness (ASTM Method D 4366-95-Method A)=126 seconds

Pencil Hardness (ASTM Method D 3363)=1H

One-Eight Inch, Conical Mandrel Bend (ASTM Method D 522-93a)=88 mm Failure

Cross Hatch Adhesion (ASTM Method 3359)=5B Rating (No Failure)

Methyl Ethyl Ketone Double Rubs (ASTM Method D 4752-87)=>200

Direct and Reverse Impact Strength (ASTM Method D2794)=48 and <4 inch pounds

A portion of the mixture comprising the diglycidyl ether of bisphenol A, the triethylenetetramine adduct, and BYK-310 was applied to 4 Tru Aluminum unpolished, coil coat white panels (3 inches×6 inches×0.038 inch) from ACT Laboratories. The coatings obtained on these panels were also cured 2 days at 60° C. After curing, the gloss of the coatings was measured using a gloss meter according to ASTM method D-523. The average gloss (percent light reflectance) at angles of 20°, 60° and 85° for the coated panels were 4.1 percent, 21.9 percent and 33.7 percent, respectively.

A portion of the mixture comprising the diglycidyl ether of bisphenol A, the triethylenetetramine adduct, and BYK-310 was also applied to two glass drying strips from which the following results were obtained using a B-K Drying Recorder manufactured by Paul N. Gardner Company, Inc.:

Set to touch time=2.0 hours

Dust free time=3.3 hours

Dry-through time=>24 hours

TABLE 1
Properties of Cured Coatings on Cold-Roll Steel Panels

| | Coating | ⅛-inch | Cross Hatch | Impact Resistance | | Pendulum | MEK |
|---|---|---|---|---|---|---|---|
| Number/ | Thickness | Mandrel | Adhesion | (Direct/Reverse) | Pencil | Hardness | Double |
| Letter | (mils) | Bend | Rating | in. lbs. | Hardness | (sec.) | Rubs |
| 3 | 2.68 | No Failure | 3B | 104/16 | 3H | 180 | >200 |
| 4 | 3.18 | Delaminated | 1B | 64/8 | 3H | 185 | >200 |
| A | 1.89 | Delaminated | 0B | 52/<4 | 4H | 198 | >200 |
| B | 1.84 | No Failure | 4B | 160/12 | 2H | 188 | >200 |
| C | 1.63 | 39 mm | 4B | 76/<4 | 4H | 160 | >200 |
| 8 | 3.37 | 100 mm | 5B | 84/4 | 1H | 65 | >200 |
| 9 | 1.02 | No Failure | 3B | >160/>160 | <4B | 37 | >200 |
| 10 | 1.66 | No Failure | 5B | 148/80 | 1H | 166 | >200 |
| 11 | 2.53 | 88 mm | 5B | 48/<4 | 1H | 126 | >200 |

TABLE 2
B-K Drying Time Test Results

| Number/ | Set to Touch Time | Dust Free Time | Dry-Through Time |
|---|---|---|---|
| Letter | (hours) | (hours) | (hours) |
| 3 | 1.9 | 2.25 | 3.4 |
| 4 | 5 | 6.25 | 11.5 |
| A | 3.6 | 5.6 | 8.1 |
| B | 3.9 | 8.1 | 12.75 |
| C | 4.0 | 4.5 | 9.2 |
| 8 | 1.7 | 2.8 | 17.8 |
| 9 | 1.6 | 2.8 | 21.7 |
| 10 | 1.6 | 1.9 | 4.7 |
| 11 | 2.0 | 3.3 | >24 |

TABLE 3
Spectral Gloss after Exposure to Conditions of QUVA Chamber

| | Exposure | 20°, | 60°, | 85°, |
|---|---|---|---|---|
| Number/ | Time | measured | measured | measured |
| Letter | (hours) | (decrease) | (decrease) | (decrease) |
| 3 | 0 | 89.6 | 99.3 | 100.0 |
| 3 | 337 | 62.4 (−30.4%) | 81.8 (−17.6%) | 97.3 (−2.7%) |
| 3 | 498 | 1.3 (−98.5%) | 7.5 (−92.4%) | 47.8 (−52.2%) |
| 4 | 0 | 96.1 | 98.2 | 99.6 |
| 4 | 337 | 2 (−97.9%) | 16.2 (−83.5%) | 48.4 (−51.4%) |
| 4 | 498 | 1.3 (−98.6%) | 7.2 (−92.8%) | 79.1 (−79.1%) |
| A | 0 | 17.8 | 44.4 | 76.7 |
| A | 288 | 1.1 (−93.8%) | 4.1 (−90.8%) | 28.0 (−63.5%) |
| A | 449 | 1.0 (−94.4%) | 3.5 (−92.1%) | 17.7 (−76.9%) |
| B | 0 | 93.3 | 100.0 | 100.0 |
| B | 288 | 22.7 (−75.7%) | 29.3 (−70.7%) | 26.2 (−73.8%) |
| B | 449 | 1.1 (−98.8%) | 5.2 (−94.8%) | 20.2 (−79.8%) |
| C | 0 | 27.9 | 55.4 | 88.7 |
| C | 71 | 19.2 (−31.2%) | 53.0 (−4.3%) | 84.8 (−4.4%) |

Advantageously, embodiments disclosed herein may provide for one or more of: lower viscosities, which may eliminate the need for solvents in coatings formulations (no VOC's); excellent UV stability in combination with good adhesion and corrosion resistance, which may eliminate the need for multiple coats in many industrial, marine, and automotive applications; and improved flexibility and damage tolerance for epoxy resin coatings. Additionally, compositions described herein may have higher crosslink density (improved thermal stability), improved reactivity due to the structural design of the backbone, higher degrees of epoxidation (fewer side-products), and glycidyl ether functionality.

1. An adduct comprising at least one reaction product of an epoxy resin material (A) and a compound (B); wherein the epoxy resin material (A) comprises a glycidyl ether of an alkanolamide or a glycidyl ester of an alkanolamide; and wherein compound (B) comprises a compound having two or more reactive hydrogen atoms per molecule, and the reactive hydrogen atoms are reactive with epoxide groups.

2. The adduct according to claim 1, wherein the alkanolamide is a seed oil based alkanolamide or a non-seed oil based alkanolamide.

3. The adduct according to claim 1, wherein the compound (B) comprises at least one of (a) a di- or a polyphenol, (b) a di- or a polycarboxylic acid, (c) a di- or a polymercaptan, (d) a di- or a polyamine, (e) a primary monoamine, (f) a-sulfonamide, (g) an aminophenol, (h) an aminocarboxylic acid, (i) a phenolic hydroxyl containing carboxylic acid, (j) a sulfanilamide, and (k) any combination thereof.

4. The adduct according to claim 3, wherein compound (B) comprises an aliphatic or cycloaliphatic diamine, an aliphatic or cycloaliphatic polyamine, or any combination thereof.

5. The adduct according to claim 1, wherein the ratio of compound (B) to the epoxy resin material (A) is 2:1 to 100:1 equivalents of the reactive hydrogen atoms in the compound (B) per equivalent of epoxide group in the epoxy resin material (A).

6. A process for preparing an adduct comprising the step of reacting at least one of an epoxy resin material (A) and a compound (B), wherein the epoxy resin material (A) comprises a glycidyl ether or ester of an alkanolamide based on at least one of a fatty acid ester, fatty acid and a fatty acid triglyceride, and the compound (B) comprises a compound having two or more reactive hydrogen atoms per molecule, and the reactive hydrogen atoms are reactive with epoxide groups.

7. The process according to claim 6, wherein the process is conducted at a temperature of from about 0° C. to about 260° C.

8. The process according to claim 6, wherein the epoxy resin material (A) is (i) directly mixed together with compound (B), (ii) added to compound (B) in incremental steps, or (iii) added to compound (B) continuously.

9. The process according to claim 6, wherein the epoxy resin material (A) further comprises at least one solvent; and/or wherein compound (B) further comprises at least one solvent.

10. The adduct according to claim 1, wherein the at least one reaction product includes an epoxy resin compound (C), wherein the resin compound (C) comprises one or more epoxy resins other than the epoxy resin material (A).

11. The process according to claim 6, wherein the step of reacting at least one reaction product includes an epoxy resin compound (C), wherein the resin compound (C) comprises one or more epoxy resins other than the epoxy resin material (A).

12. A curable epoxy resin composition comprising (a) an adduct of claim 1 or claim 10, and (b) a resin compound (D); wherein the resin compound (D) comprises one or more epoxy resins other than the epoxy resin material (A) and other than the epoxy resin (C).

13. The composition according to claim 12, wherein the compound (B) in the adduct comprises an aliphatic or cycloaliphatic diamine, an aliphatic or cycloaliphatic polyamine, or any combination thereof.

14. The composition according to claim 12, wherein the ratio of the adduct and the resin compound (D) is 0.60:1 to 1.50:1 equivalents of reactive hydrogen atom in the adduct per equivalent of epoxide group in the resin compound (D).

15. The composition according to claim 12 further comprising a curing agent; a curing catalyst; or a linear chain extender.

16. The composition according to claim 15, wherein the linear chain extender is a reaction product of the epoxy resin material (A) and the material (B); wherein and the epoxy resin material (A) comprises a glycidyl ether or ester of at least one of an alkanolamides, a saturated fatty acid ester, and a fatty acid triglyceride; and wherein the material (B) comprises a primary monoamine or a secondary diamine.

17. A process for curing the curable epoxy resin composition of claim 12, wherein the process is conducted at temperature of from about 0° C. to about 300° C.

18. The process according to claim 17, wherein the process is partially cured at a B-stage and then completely cured at a later time.

19. An article comprising the cured epoxy resin prepared by the process of curing the curable epoxy resin composition of claim 12; and wherein the article is at least one of a coating, an electrical or structural laminate, an electrical or structural composite, a filament winding, a molding, a casting, and an encapsulation.
